(19) United States
(12) Patent Application Publication
(10) Pub. No.: US 2023/0172870 A1
(43) Pub. Date: Jun. 8, 2023

(54) SURFACE CONJUGATION TO POLY(AMINE-CO-ESTER) NANOPARTICLES FOR TARGETING TO CELLS AND TISSUES

(71) Applicant: Yale University, New Haven, CT (US)

(72) Inventors: Alexandra Piotrowski-Daspit, Wethersfield, CT (US); Claire Albert, New Haven, CT (US); W. Mark Saltzman, New Haven, CT (US)

(21) Appl. No.: 18/063,613

(22) Filed: Dec. 8, 2022

Related U.S. Application Data
(60) Provisional application No. 63/287,410, filed on Dec. 8, 2021.
(60) Provisional application No. 63/290,042, filed on Dec. 15, 2021.
(60) Provisional application No. 63/292,200, filed on Dec. 21, 2021.
(60) Provisional application No. 63/301,942, filed on Jan. 21, 2022.
(60) Provisional application No. 63/302,413, filed on Jan. 24, 2022.
(60) Provisional application No. 63/418,744, filed on Oct. 24, 2022.

(51) Int. Cl. A61K 9/51; A61K 47/60; A61K 47/34

(52) U.S. Cl. CPC A61K 9/5153; A61K 47/60; A61K 47/34; B82Y 5/00

(57) ABSTRACT

Nanoparticles useful for drug delivery are described. In one aspect, the nanoparticles contain poly(amine-co-ester)s or poly(amine-co-amide)s (PACE) modified with poly(ethylene glycol) (PACE-PEG), and can be optionally blended with a second PACE polymer optionally containing endgroup modifications. In another aspect, the nanoparticles contain a core containing a PACE polymer optionally containing endgroup modifications, and a polymeric surfactant non-covalently conjugated to the surface of the nanoparticles. The nanoparticles contain a peptide or protein targeting moiety that is covalently conjugated to the PACE-PEG polymer or to the surfactant on the surface of the nanoparticles via a linkage that contains a succinimide or substituted sulfone moiety, respectively. The nanoparticles provide as a versatile platform for the delivery of nucleic acids, such as mRNA.

### CROSS REFERENCE TO RELATED APPLICATIONS

This application claims the benefit of and priority to U.S. Ser. No. 63/287,410 filed Dec. 8, 2021; U.S. Ser. No. 63/290,042, filed Dec. 15, 2021; U.S. Ser. No. 63/292,200, filed Dec. 21, 2021; U.S. Ser. No. 63/301,942 filed Jan. 21, 2022; U.S. Ser. No. 63/302,413, filed Jan. 24, 2022; and U.S. Ser. No. 63/418,744, filed Oct. 24, 2022; which are specifically incorporated by reference herein in their entirety.

### STATEMENT REGARDING FEDERALLY SPONSORED RESEARCH

This invention was made with government support under HL147352, AI32895, GM86287, and HL142144 awarded by National Institutes of Health. The government has certain rights in the invention.

### FIELD OF THE INVENTION

The field of the invention is generally related to polymer compositions and methods for improved conjugation of molecules (e.g., peptides and proteins) to polymeric nanoparticles, for efficient delivery of diagnostic, prophylactic, and/or therapeutic agents, including nucleic acids (e.g., mRNA).

### BACKGROUND OF THE INVENTION

Non-viral vectors for gene delivery have attracted much attention in the past several decades due to their potential for limited immunogenicity, ability to accommodate and deliver large size genetic materials, and potential for modification of their surface structures. Major categories of non-viral vectors include cationic lipids and cationic polymers. Cationic lipid-derived vectors, which were pioneered by Felgner and colleagues, represent some of the most extensively investigated systems for non-viral gene delivery (Felgner, et al. PNAS, 84, 7413-7417 (1987)) (Templeton, et al. Nat. Biotechnol. 15, 647-652 (1997)) (Chen, et al. J. Invest. Dermatol. 130, 2790-2798 (2010)).

Cationic polymer non-viral vectors have gained increasing attention because of flexibility in their synthesis and structural modifications for specific biomedical applications. Both cationic lipid and cationic polymer systems deliver genes by forming condensed complexes with negatively charged DNA through electrostatic interactions: complex formation protects DNA from degradation and facilitates its cellular uptake and intracellular traffic into the nucleus.

Polyplexes formed between cationic polymers and DNA are generally more stable than lipoplexes formed between cationic lipids and DNA, but both are often unstable in physiological fluids, which contain serum components and salts, and tend to cause the complexes to break apart or aggregate (Al-Dosari, et al. AAPS J. 11, 671-681 (2009)) (Tros de Ilarduya, et al. Eur. J. Pharm. Sci. 40, 159-170 (2010)). Additionally, although some work indicates that anionic polymers or even naked DNA can provide some level of transfection under certain conditions, transfection by both lipids and polymers usually requires materials with excess charge, resulting in polyplexes or lipoplexes with net positive charges on the surface (Nicol, et al. Gene. Ther. 9, 1351-1358 (2002)) (Schlegel, et al. J. Contr. Rel. 152, 393-401 (2011)) (Liu, et al, AAPS J. 9, E92-E104 (2007)) (Liu, et al. Gene Ther. 6, 1258-1266 (1999)). When injected into the circulatory system in vivo, the positive surface charge initiates rapid formation of complex aggregates with negatively charged serum molecules or membranes of cellular components, which are then cleared by the reticuloendothelial system (RES).

More importantly, many cationic vectors developed so far exhibit substantial toxicity, which has limited their clinical applicability (Tros de Ilarduya, et al. Eur. J. Pharm. Sci. 40, 159-170 (2010)) (Gao, et al. Biomaterials 32, 8613-8625 (2011)) (Feigner, et al. J. Biol. Chem. 269, 2550-2561 (1994)) (Kafil, et al. BioImpacts 1, 23-30 (2011)) (Lv, et al. J Contr. Rel. 114, 100-109 (2006)). This too appears to depend on charge: excess positive charges on the surface of the complexes can interact with cellular components, such as cell membranes, and inhibit normal cellular processes, such as clathrin-mediated endocytosis, activity of ion channels, membrane receptors, and enzymes or cell survival signaling (Gao, et al. Biomaterials 32, 8613-8625 (2011)) (Feigner, et al. Enhanced gene delivery and mechanism studies with a novel series of cationic lipid formulations. J. Biol. Chem. 269, 2550-2561 (1994)) (Kafil, et al. Cytotoxic Impacts of Linear and Branched Polyethylenimine Nanostructures in A431 Cells. BioImpacts 1, 23-30 (2011)).

As a result, cationic lipids often cause acute inflammatory responses in animals and humans, whereas cationic polymers, such as PEI, destabilize the plasma-membrane of red blood cells and induce cell necrosis, apoptosis and autophagy (Tros de Ilarduya, et al. Eur. J. Pharm. Sci. 40, 159-170 (2010)) (Gao, et al. Biomaterials 32, 8613-8625 (2011)) (Lv, et al. J. Contr. Rel. 114, 100-109 (2006)). Because of these undesirable effects, there is a need for highly efficient non-viral vectors that have lower charge densities.

Synthesis of a family of biodegradable poly(amine-co-esters) formed via enzymatic copolymerization of diesters with amino-substituted diols is discussed in Liu, et al. J. Biomed. Mater. Res. A 96A, 456-465 (2011) and Jiang, Z. Biomacromolecules 11, 1089-1093 (2010). Diesters with various chain length (e.g., from succinate to dodecanedioate) were copolymerized with diethanolamines with either an alkyl (methyl, ethyl, n-butyl, t-butyl) or an aryl (phenyl) substituent on the nitrogen. The high tolerance of the lipase catalyst allowed the copolymerization reactions to complete in one step without protection and deprotection of the amino functional groups. Upon protonation at slightly acidic conditions, these poly(amine-co-esters) readily condense DNA and form nano-sized polyplexes. Screening studies revealed that one of these materials, poly(N-methyldiethyleneamine sebacate) (PMSC), transfected a variety of cells including HEK293, U87-MG, and 9L, with efficiency comparable to that of leading commercial products, such as Lipofectamine 2000 and PEI14. PMSC had been previously used for gene delivery, but the delivery efficiency of the enzymatically synthesized materials was approximately five orders of magnitude higher than any previously reported (Wang, et al. Biomacromolecules 8, 1028-1037 (2007)) (Wang, et al. Biomaterials 28, 5358-5368 (2007)). However, these poly(amine-co-esters) were not effective for systemic delivery of nucleic acids in vivo. To address these limitations, a safe and effective nucleic acid delivery platform is needed.

Therefore, it is an object of the invention to provide an effective, nontoxic, and sustained release delivery system for nucleic acids such as messenger ribonucleic acids (mRNA).

It is also an object of the invention to provide targeted polymeric nanoparticles for an effective, nontoxic, and sustained release delivery system for nucleic acids such as mRNA.

### SUMMARY OF THE INVENTION

Nanoparticles that are especially useful for targeted drug delivery are described. In some forms, the nanoparticles contain poly(amine-co-ester)s or poly(amine-co-amide)s (PACE) modified with poly poly(ethylene glycol) (PACE-PEG). The nanoparticles can also contain a blend of the PACE-PEG polymer with a second PACE polymer that polymer optionally contains an endgroup modification. A PACE polymer having an endgroup modification is denoted (PACEng).

In some forms, the PACE-PEG polymer contains a peptide or protein targeting moiety, covalently conjugated thereto through a linkage that preferably contains a succinimide.

In some forms, the nanoparticles contain a surfactant non-covalently conjugated to the surface of the nanoparticles. Preferably, the surfactant is a polymer, such as a polymer formed from a poly(vinyl alcohol sulfone) polymer. In these forms, the nanoparticles contain a PACE polymer that optionally contains an endgroup modification. In these forms, the surfactant contains a peptide or protein is covalently conjugated to the surfactant via a linkage that preferably contains a substituted sulfone moiety.

The PACE polymers whether PEG-modified or endgroup modified, contain lactone, polyfunctional molecule, and diacid/diester monomeric units. Preferably, in some forms of the PACEng polymer, the percent composition of the lactone unit is between about 30% and about 100%, calculated lactone unit vs. (lactone unit+diester/diacid). Expressed in terms of a molar ratio, the lactone unit vs. (lactone unit+diester/diacid) content is between about 0.3 and about 1, i.e., x/(x+q) is between about 0.3 and about 1.

The nanoparticles can be used as a versatile platform for the delivery of nucleic acids, such as mRNA.

### BRIEF DESCRIPTION OF THE DRAWINGS

FIGS. 1A and 1B are nuclear magnetic resonance (NMR) spectra confirming the presence of PEG-Maleimide in structure of PACE-PEG-Maleimide (PACE-PEG-MAL). FIG. 1A, from Kamaly, et al., Proc. Natl. Acad. Sci., USA shows the NMR spectrum of diblock poly(lactic-co-glycolic acid)-b-poly(ethylene glycol)-Maleimide (PLGA-PEG-MAL), including a chemical structure which shows the assignment of each of the protons in the structure to peaks in the NMR spectrum. Importantly the peaks for the maleimide group appear at 6.7 ppm. FIG. 1B shows the NMR spectrum of PACE-PEG-Maleimide. This spectrum confirms the introduction of a maleimide group (see the peak at 6.7 ppm) to the PACE-PEG polymer.

FIG. 2A is a schematic showing the conjugation (e.g., covalent conjugation) of a protein to the surface of nanoparticles via PACE-PEG-MAL. FIG. 2B is a line graph showing enhanced targeted uptake by cells treated in vitro with dye-loaded PEG-PEG-MAL nanoparticles functionalized with antibodies. The cells are human bronchial epithelial cells homozygous for the W1282X nonsense cystic fibrosis mutation. The antibodies target the epithelial marker EpCAM (right-shifted histogram) or are isotype control antibodies (left-shifted histogram).

FIG. 3A is a schematic showing incubation of protein-conjugated nanoparticles via PACE-PEG-MAL on the nanoparticles. FIG. 3B is a column graph showing increased targeted uptake by human umbilical vein endothelial cells (HUVEC) of PEG-PEG-MAL nanoparticles functionalized with CD31.

FIG. 4A is a schematic showing the chemical synthesis of poly(vinyl alcohol-vinyl sulfone) copolymer (PVA-VS) with some vinyl acetate monomers present. FIGS. 4C and 4C are NMR spectra confirming the structure of PVA-VS.

FIG. 5A is a schematic showing the chemical synthesis of PVA-VS with no vinyl acetate monomers present. FIG. 5B is a line graph showing representative flow cytometry histograms of uptake by HUVECs treated in vitro with PACE-PVA-VS-binding protein (CD31-Mb) nanoparticles, PACE-PVA-VS-control (isotype-Mb) nanoparticles, or no nanoparticles. FIG. 5C is a column graph of the mean fluorescence intensity (MFI) values of fluorescent binding protein-PACE-NP bound to HUVECs in vitro (error bars represent SD). Each data point represents the averaged from 3 wells (10,000 HUVECs captured/well). Statistical significance is shown between groups with “****” where p<0.001, “***” where p<0.001, “**” where p<0.01, “*” where p<0.05 and “ns” where no significant difference was found using multiple T-tests with a Bonferroni correction.

FIG. 6A-6F are a schematic of PACE-PEG-MAL NP monobody and antibody conjugation process (FIG. 6A); a graph of the % NP+ CD4⁺ PBMCs from human blood following in vitro incubation with either isotype control or CD4-targeting DiD-loaded PACE-PEG-MAL NPs for 5 min, 12 min, 30 min, and 1 hr. (FIG. 6B); a graph of the % NP+ PBMCs from human blood following in vitro incubation with either isotype control or CD4-targeting DiD-loaded PACE-PEG-MAL NPs for 5 min, 12 min, 30 min, and 1 hr (FIG. 6C); a schematic of in vivo CD4⁺ T-cell PACE-PEG-MAL NP targeting experiment with clodronate liposome decoy pre-treatment in rats (FIG. 6D; graphs of the % NP+ CD4⁺ PBMCs from rat blood two hours after intravenous delivery in vivo of either isotype control or CD4-targeting DiD-loaded PACE-PEG-MAL NPs (FIG. 6E); and graphs of % NP+ PBMCs from rat blood two hours after intravenous delivery in vivo of either isotype control or CD4-targeting DiD-loaded PACE-PEG-MAL NPs (FIG. 6F).

### DETAILED DESCRIPTION OF THE INVENTION

### I. Definitions

Nanoparticles are particles (nanospheres and nanocapsules) with diameters between 1 nm and less than 1 micron. A nanoparticle may be spherical or nonspherical and may have a regular or irregular shape. In certain embodiments, populations of the nanoparticles have an average diameter of about 500 nm, 200 nm, 100 nm, 50 nm, or 10 nm. In some embodiments, the average diameter of the particles is from about 100 nm to about 600 nm, about 100 nm to 500 nm, about 100 nm to 300 nm, about 100 nm to 250 nm, about 200 nm to 500 nm, about 200 nm to 400 nm, or about 200 nm to 350 nm. In some embodiments, the average diameter is from about 100 nm to 250 nm. In some embodiments, the average diameter is from about 200 nm to 350 nm. preferably from about 200 to about 500 nm. The term “diameter” is used herein to refer to either of the physical diameter or the hydrodynamic diameter. The diameter of an essentially spherical particle may refer to the physical or hydrodynamic diameter. The diameter of a non-spherical particle may refer preferentially to the hydrodynamic diameter. The diameter of a non-spherical particle may refer to the largest linear distance between two points on the surface of the particle. When referring to multiple particles, the diameter of the particles typically refers to the average diameter of the particles. Particle diameter can be measured using a variety of techniques in the art including, but not limited to, dynamic light scattering.

The term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

The term “biodegradable” as used herein means that the material degrades or breaks down into its component subunits, or digestion, e.g., by a biochemical process, of the material into smaller (e.g., non-polymeric) subunits.

The phrase “sustained release” refers to release of a substance over an extended period of time, in contrast to a bolus type administration in which the majority of the substance is made biologically available at one time.

The phrases “parenteral administration” and “administered parenterally” are art-recognized terms, and include modes of administration other than enteral and topical administration, typically by injection, and can include intravenous, intramuscular, intrapleural, intravascular, intradermal, intraperitoneal, transtracheal, and subcutaneous injection and infusion.

The term “surfactant” as used herein refers to an agent that lowers the surface tension of a liquid.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The terms “lactone” and “lactone unit” are used to describe a chemical compound that includes a cyclic ester, or the open chain chemical structure that results from the cleavage of the ester bond in the cyclic ester. For example, lactone is used to describe the cyclic ester shown below, and the corresponding lactone-derived open chain structure:

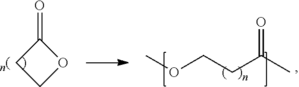

n being an integer. The open chain structure is formed via methods known in the art, including but not limited to, solvolysis, such as hydrolysis, and enzymatic cleavage.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In preferred embodiments, a straight chain or branched chain alkyl has 30 or fewer carbon atoms in its backbone (e.g., C1-C30 for straight chains, C3-C30 for branched chains), preferably 20 or fewer, more preferably 15 or fewer, most preferably 10 or fewer. All integer values of the number of backbone carbon atoms between one and 30 are contemplated and disclosed for the straight chain or branched chain alkyls. Likewise, preferred cycloalkyls have from 3-10 carbon atoms in their ring structure, and more preferably have 5, 6, or 7 carbons in the ring structure. All integer values of the number of ring carbon atoms between three and 10 are contemplated and disclosed for the cycloalkyls.

The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

It will be understood by those skilled in the art that the moieties substituted on the hydrocarbon chain can themselves be substituted, if appropriate. For example, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like. Cycloalkyls can be substituted in the same manner.

“Aryl” refers to C5-C10-membered aromatic, heterocyclic, fused aromatic, fused heterocyclic, biaromatic, or bihetereocyclic ring systems. In some forms, the ring systems have 3-50 carbon atoms. Broadly defined, “aryl”, as used herein, includes 5-, 6-, 7-, 8-, 9-, 10- and 24-membered single-ring aromatic groups that may include from zero to four heteroatoms, for example, benzene, naphthalene, anthracene, phenanthrene, chrysene, pyrene, corannulene, coronene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

“Alkoxy” refers to an alkyl group as defined above with the indicated number of carbon atoms attached through an oxygen bridge. Examples of alkoxy include, but not limited to, methoxy, ethoxy, n-propoxy, i-propoxy, n-butoxy, s-butoxy, n-pentoxy, s-pentoxy, and derivatives thereof.

Primary amines arise when one of three hydrogen atoms in ammonia is replaced by a substituted or unsubstituted alkyl or a substituted or unsubstituted aryl group. Secondary amines have two organic substituents (substituted or unsubstituted alkyl, substituted or unsubstituted aryl or combinations thereof) bound to the nitrogen together with one hydrogen. In tertiary amines, nitrogen has three organic substituents.

“Substituted”, as used herein, means one or more atoms or groups of atoms on the monomer has been replaced with one or more atoms or groups of atoms which are different than the atom or group of atoms being replaced. In some embodiments, the one or more hydrogens on the monomer is replaced with one or more atoms or groups of atoms. Examples of functional groups which can replace hydrogen are listed above in the definition. In some embodiments, one or more functional groups can be added which vary the chemical and/or physical property of the resulting monomer/polymer, such as charge or hydrophilicity/hydrophobicity, etc. Exemplary substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The terms “inhibit” or “reduce” generally mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%, or an integer there between. In some embodiments, the inhibition and reduction are compared at mRNA, protein, cellular, tissue and/or organ levels.

The terms “prevent”, “prevention” or “preventing” mean to administer a composition or method to a subject at risk for, or having a predisposition for, one or more symptom caused by a disease or disorder, to decrease the likelihood the subject will develop one or more symptoms of the disease or disorder, or to reduce the severity, duration, or time of onset of one or more symptoms of the disease or disorder.

### II. Compositions

A. Nanoparticles Containing poly(amine-co-ester)s and/or poly(amine-co-amide)s Blended with and/or Modified with Polyalkylene Oxide

Nanoparticles useful for the targeted delivery of therapeutic, diagnostic, and/or prophylactic agents are disclosed. Preferably, the agents are nucleic acids, such as mRNA.

1. Poly(amine-co-ester)s and/or poly(amine-co-amide)s Modified with Polyalkylene Oxide

In one aspect, the nanoparticles contain poly(amine-co-ester)s and/or poly(amine-co-amide)s (PACE) modified with a polyalkylene oxide (PACE-PAO). The PAO contains the moiety [O-Cna-O]—, wherein na is at least 2, such as between 2 and 8, inclusive, preferably between 2 and 3, inclusive. The carbon atoms can be independently unsubstituted or substituted, such as substituted with hydroxyl groups, carbinol groups, or a combination thereof. For illustrative purposes, when na is 2 and the carbon atoms are unsubstituted, the PAO is poly(ethylene glycol); when na is 2, and at least one of the carbon atoms are independently substituted with a carbinol moiety, the PAO is poly(glycerol); and when na is 2 and/or 3, and at least one of the carbon atoms are independently substituted with hydroxyl and/or carbinol groups, the PAO is hyperbranched polyglycerol. Preferably, the polyalkylene oxide is poly(ethylene glycol) (PEG). The PACE-PAO polymer is optionally blended with a second PACE polymer that optionally contains an endgroup modification.

In some forms, the nanoparticles contain between about 0.1% wt/wt and about 95% wt/wt, between about 0.1% wt/wt and about 50% wt/wt, between about 0.1% wt/wt and about 20% wt/wt, between about 0.1% wt/wt and about 10% wt/wt, between about 0.1% wt/wt and about 5% wt/wt, between about 1% wt/wt and about 5% wt/wt, between 5% wt/wt and 10% wt/wt, or between about 10% wt/wt and 20% about wt/wt of PACE-PAO, such as PACE-PEG, expressed in terms of weight of the PACE-PAO polymer to total weight of the nanoparticles.

The PACE-PAO polymers have the general formula:

((A)ₓ-(B)_{y}-(C)_{q}-(D)_{w}-(E)_{f})ₕ, Formula Ia′

wherein A, B, C, D, and E independently include monomeric units derived from lactones (such as pentadecalactone), a polyfunctional molecule (such as N-methyldiethanolamine), a diacid or diester (such as diethylsebacate), or polyalkylene oxide (such as polyethylene glycol), wherein the PACE-PAO polymers include at least a lactone, a polyfunctional molecule, a diacid or diester monomeric units, and polyalkylene oxide (such as polyethylene glycol). In general, the polyfunctional molecule contains one or more cations, one or more positively ionizable atoms, or combinations thereof. The one or more cations are formed from the protonation of a basic nitrogen atom, or from quaternary nitrogen atoms.

In general, for the PACE-PAO polymers, x, y, q, w, and f are independently integers from 0-1000, with the proviso that the sum (x+y+q+w+f) is greater than one. h is an integer from 1 to 1000.

In some forms of the PACE-PAO polymers, the percent composition of the lactone unit is between about 10% and about 100%, calculated lactone unit versus (lactone unit+diester/diacid). Expressed in terms of a molar ratio, the lactone unit versus (lactone unit+diester/diacid) content is between about 0.1 and about 1, i.e., x/(x+q) is between about 0.1 and about 1. Preferably, in some forms of the PACE-PAO polymers, the percent composition of the lactone unit is between about 30% and about 100%, calculated lactone unit versus (lactone unit+diester/diacid). Expressed in terms of a molar ratio, the lactone unit vs. (lactone unit+diester/diacid) content is between about 0.3 and about 1, i.e., x/(x+q) is between about 0.3 and about 1. Preferably, the number of carbon atoms in the lactone unit is between about 10 and about 24, more preferably the number of carbon atoms in the lactone unit is between about 12 and about 16. Most preferably, the number of carbon atoms in the lactone unit is 12 (dodecalactone), 15 (pentadecalactone), or 16 (hexadecalactone).

The structure of a PACE-PEG-containing polymer is shown below:

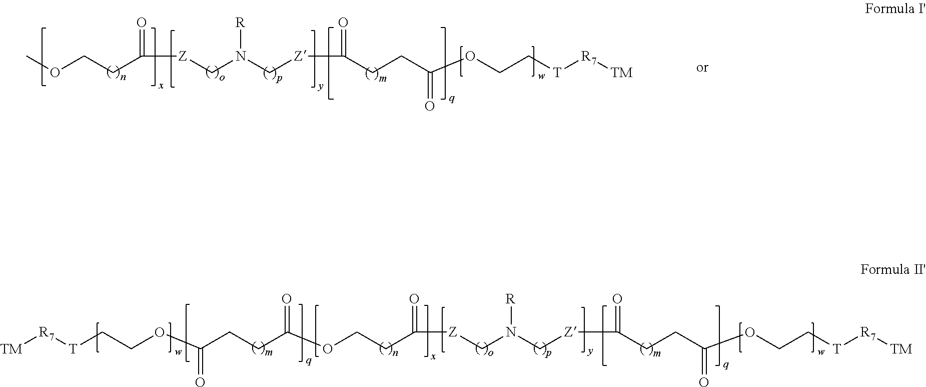

preferably Formula I′,

wherein n is an integer from 1-30, m, o, and p are independently an integer from 1-20, x, y, and q, are independently integers from 1-1000, w is independently an integer from 0-1000, with the proviso that in Formula I′, w is greater than 0, and in Formula II′, at least one w is not zero (0),

Z and Z′ are independently 0 or NR′, wherein R and R′ are independently hydrogen, substituted or unsubstituted alkyl, or substituted or unsubstituted aryl,

wherein T is independently absent, oxygen, sulfur, alkyl, substituted alkyl, amide, substituted amide, amine, substituted amine, carbonyl, substituted carbonyl,

wherein R₇ is independently hydrogen, alkyl, substituted alkyl, amide, substituted amide, substituted sulfone, unsubstituted sulfone, aryl, substituted aryl, cycloalkyl, substituted cycloalkyl, maleimide, amine, substituted amine, thiol, N-hydroxysuccinimide ester, succinimide, azide, acrylate, methacrylate, alkyne, hydroxide, or isocynate, and

TM is independently absent or a targeting moiety.

In some forms of the PACE-PEG polymer, Z is the same as Z′ in Formula I′ or Formula II′.

In some forms of the PACE-PEG polymer, Z is O and Z′ is O. In some forms of the PACE-PEG polymer, Z is NR′ and Z′ is NR′ in Formula I′ or Formula II′. In some forms of the PACE-PEG polymer, Z is O and Z′ is NR′ in Formula I′ or Formula II′. In some forms of the PACE-PEG polymer, Z is NR′ and Z′ is O in Formula I′ or Formula II′.

In some forms of the PACE-PEG polymer, Z′ is O and n is an integer from 1-24, such as 4, 10, 13, or 14. In some forms, Z is also O in Formula I′ or Formula II′.

In some forms of the PACE-PEG polymer, Z′ is O, n is an integer from 1-24, such as 4, 10, 13, or 14, and m is an integer from 1-10, such as 4, 5, 6, 7, or 8. In some forms, Z is also O in Formula I′ or Formula II′.

In some forms of the PACE-PEG polymer, Z′ is O, n is an integer from 1-24, such as 4, 10, 13, or 14, m is an integer from 1-10, such as 4, 5, 6, 7, or 8, and o and p are the same integer from 1-6, such 2, 3, or 4 in Formula I′ or Formula II′. In some forms, Z is also O in Formula I′ or Formula II′.

In some forms of the PACE-PEG polymer, Z′ is O, n is an integer from 1-24, such as 4, 10, 13, or 14, m is an integer from 1-10, such as 4, 5, 6, 7, or 8, and R is alkyl, such a methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, and n-octyl, or aryl, such as phenyl, naphthalyl, anthracenyl, phenanthryl, chrysenyl, pyrenyl, tolyl, or xylyl in Formula I′ or Formula II′. In some forms, Z is also O in Formula I′ or Formula II′.

In some forms of the PACE-PEG polymer, n is 13 (e.g., pentadecalactone, PDL), m is 7 (e.g., sebacic acid), o and p are 2 (e.g., N-methyldiethanolamine, MDEA) in Formula I′ or Formula II′.

In particular embodiments, the values of x, y, q, and w are such that the weight average molecular weight of the PACE-PEG polymer is greater than 5,000 Daltons, such as between 5,000 Daltons and 50 kDa, between 5,000 Daltons and 30 kDa in Formula I′ or Formula II′. This weight average molecular weight does not include the molecular weight of the targeting moiety. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

The blocks of polyalkylene oxide can located at the termini of the polymer (i.e., by reacting PEG having one hydroxy group blocked, for example, with a methoxy group), within the polymer backbone (i.e., neither of the hydroxyl groups are blocked), or combinations thereof.

In some forms, the nanoparticles contain PACE-PEG polymers of Formula I′ and/or Formula II′, as described above, wherein at least a targeting moiety (TM) is present in Formula I and/or Formula II′. Preferably, covalent conjugation occurs through T, R₇, or a combination thereof. In some forms, such as when the targeting moiety is a protein or peptide, conjugation proceeds via a facile “click” reaction between a reactive group on the protein or peptide (e.g., a thiol) and a maleimide group on the polymer to form a succinimide group linking the targeting moiety to the PEG terminal of the polymer.

2. Nanoparticles Containing PACE-PAO Blended with a Second PACE Polymer

In some forms, the nanoparticles contain a blend of a PACE-PAO polymer (as described above under the section titled Nanoparticles containing PACE-PAO) and a second PACE polymer. Preferably, the second PACE polymer does not contain a PEG modification. The second PACE polymer may be the same or different from the PACE polymer segment in the PACE-PAO (such as PACE-PEG) polymer, where similarities or differences can be assessed based on weight average molecular weights, or molar percent compositions of components in the PACE polymers. The second PACE polymer optionally contains endgroup modifications. When the second PACE polymer does not contain an endgroup modification, it is denoted “second PACEab.” When at least one endgroup modification is present, the second PACE polymer is denoted “second PACEng.”

In some forms, the second PACE polymer has a structure as shown in Formula I:

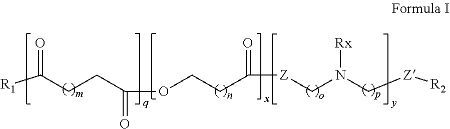

wherein n is an integer from 1-30,

m, o, and p are independently integers from 1-20,

x, y, and q are independently integers from 1-1000,

Rₓ is hydrogen, substituted or unsubstituted alkyl, or substituted or unsubstituted aryl, or substituted or unsubstituted alkoxy,

Z and Z′ are independently O or NR′, wherein R′ is hydrogen, substituted or unsubstituted alkyl, or substituted or unsubstituted aryl,

R₁ and R₂ are independently absent or are chemical entities containing a hydroxyl group, a primary amine group, a secondary amine group, a tertiary amine group, or combinations thereof.

In some forms of the second PACE polymer, R₁ and R₂ are absent. In some forms of the second PACE polymer, at least one of R₁ and R₂ is present. When R₁ and R₂ are absent, the second PACE polymer is denoted “second PACEab polymer.” When at least one of R₁ and R₂ is present, the second PACE polymer is denoted “second PACEng polymer.”

Examples of Rₓ and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

In particular embodiments, the values of x, y, and/or q are such that the weight average molecular weight of the second PACE polymer (i.e., second PACEab polymer or second PACEng polymer) is greater than 20,000 Daltons, greater than 15,000 Daltons, greater than 10,000 Daltons, greater than 5,000 Daltons, greater than 2,000 Daltons. In some forms, the weight average molecular weight of the PACEng polymer is between about 2,000 Daltons and about 30,000 Daltons, between about 2,000 Daltons and about 25,000 Daltons, between about 2,000 Daltons and about 20,000 Daltons, or between about 5,000 Daltons and about 10,000 Daltons.

The second PACE polymer (i.e., second PACEab polymer or second PACEng polymer) can be prepared from one or more lactones, one or more amine-diols (Z and Z′=O), triamines (Z and Z′=NR′), or hydroxy-diamines (Z=O and Z′=NR′, or Z=NR′ and Z′=O) and one or more diacids or diesters. In those embodiments where two or more different lactone, diacid or diester, and/or triamine, amine-diol, or hydroxy-diamine monomers are used, the values of n, o, p, and/or m can be the same or different.

In some forms of the second PACE polymer (i.e., second PACEab polymer or second PACEng polymer), the percent composition of the lactone unit is between about 10% and about 100%, calculated lactone unit vs. (lactone unit+diester/diacid). Expressed in terms of a molar ratio, the lactone unit vs. (lactone unit+diester/diacid) content is between about 0.1 and about 1, i.e., x/(x+q) is between about 0.1 and about 1. Preferably, in some forms of the second PACE polymer (i.e., second PACEab polymer or second PACEng polymer), the percent composition of the lactone unit is between about 30% and about 100%, calculated lactone unit vs. (lactone unit+diester/diacid). Expressed in terms of a molar ratio, the lactone unit vs. (lactone unit+diester/diacid) content is between about 0.3 and about 1, i.e., x/(x+q) is between about 0.3 and about 1. Preferably, the number of carbon atoms in the lactone unit is between about 10 and about 24, more preferably the number of carbon atoms in the lactone unit is between about 12 and about 16. Most preferably, the number of carbon atoms in the lactone unit is 12 (dodecalactone), 15 (pentadecalactone), or 16 (hexadecalactone).

In some forms of the second PACE polymer (i.e., second PACEab polymer or second PACEng polymer), Z is the same as Z′.

In some forms of the second PACE polymer (i.e., second PACEab polymer or second PACEng polymer), Z is O and Z′ is O. In some forms, Z is NR′ and Z′ is NR′. In some forms, Z is O and Z′ is NR′. In some forms, Z is NR′ and Z′ is O.

In some forms of the second PACE polymer (i.e., second PACEab polymer or second PACEng polymer), Z′ is O and n is an integer from 1-24, such as 4, 10, 13, or 14. In some forms, Z is also O.

In some forms of the second PACE polymer (i.e., second PACEab polymer or second PACEng polymer), Z′ is O, n is an integer from 1-24, such as 4, 10, 13, or 14, and m is an integer from 1-10, such as 4, 5, 6, 7, or 8. In some forms, Z is also O.

In some forms of the second PACE polymer (i.e., second PACEab polymer or second PACEng polymer), Z′ is O, n is an integer from 1-24, such as 4, 10, 13, or 14, m is an integer from 1-10, such as 4, 5, 6, 7, or 8, and o and p are the same integer from 1-6, such 2, 3, or 4. In some forms, Z is also O.

In some forms of the second PACE polymer (i.e., second PACEab polymer or second PACEng polymer), Z′ is O, n is an integer from 1-24, such as 4, 10, 13, or 14, m is an integer from 1-10, such as 4, 5, 6, 7, or 8, and R is alkyl, such a methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, and n-octyl, or aryl, such as phenyl, naphthalyl, anthracenyl, phenanthryl, chrysenyl, pyrenyl, tolyl, or xylyl. In some forms, Z is also O.

In some forms of the second PACE polymer (i.e., second PACEab polymer or second PACEng polymer), n is 13 (e.g., pentadecalactone, PDL), m is 7 (e.g., sebacic acid), o and p are 2 (e.g., N-methyldiethanolamine, MDEA).

In some forms, the nanoparticles contain the second PACEng polymer, wherein R₁ and/or R₂ are not relative to corresponding nanoparticles, wherein R₁ and/or R₂ consist of or include

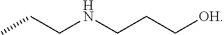

In some forms, nanoparticles containing the second PACEng polymer show improved loading, improved cellular transfection, improved intracellular endosomal release, or a combination thereof of a nucleic acid cargo, such as RNA, more particularly mRNA, relative to corresponding nanoparticles wherein R₁ and/or R₂ consist of or include

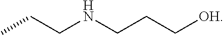

In some forms, the second PACEng polymer has a structure of Formula II:

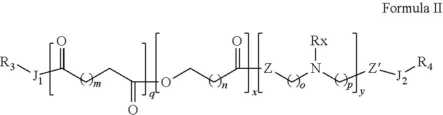

wherein J₁ and J₂ are independently linking moieties or absent,

R₃ and R₄ are independently substituted alkyl containing a hydroxyl group, a primary amine group, a secondary amine group, a tertiary amine group, or combinations thereof. In some forms, the molecular weight of R₃, R₄ or both are at or below 500 Daltons, at or below 200 Daltons, or at or below 100 Daltons.

In some forms of the second PACEng polymer, J₁ is —O— or —NH—.

In some forms of the second PACEng polymer, J₂ is —C(O)NH— or —C(O)O—.

In some forms of the second PACEng polymer, R₃ is identical to R₄.

Preferably, R₃ and/or R₄ are linear.

In some forms of the second PACEng polymer, R₃, R₄ or both contain a primary amine group. In some forms of the second PACEng polymer, R₃, R₄ or both contain a primary amine group and one or more secondary or tertiary amine groups.

In some forms of the second PACEng polymer, R₃, R₄ or both contain a hydroxyl group. In some forms of the second PACEng polymer, R₃, R₄ or both contain a hydroxyl group and one or more amine groups, preferably secondary or tertiary amine groups. In some forms of the second PACEng polymer, R₃, R₄ or both contain a hydroxyl group and no amine group.

In some forms of the second PACEng polymer, at least one of R₃ and R₄ does not contain a hydroxyl group.

In some forms of the second PACEng polymer, R₃, R₄ or both are -unsubstituted C₁-C₁₀ alkylene-Aq-unsubstituted C₁-C₁₀ alkylene-Bq, -unsubstituted C₁-C₁₀ alkylene-Aq-substituted C₁-C₁₀ alkylene-Bq, -substituted C₁-C₁₀ alkylene-Aq-unsubstituted C₁-C₁₀ alkylene-Bq, or -substituted C₁-C₁₀ alkylene-Aq-substituted C₁-C₁₀ alkylene-Bq, wherein Aq is absent or —NR₅—, and Bq is hydroxyl, primary amine, secondary amine, or tertiary amine, wherein R₅ is hydrogen, substituted or unsubstituted alkyl, or substituted or unsubstituted aryl.

In some forms of the second PACEng polymer, R₃, R₄, or both are selected from

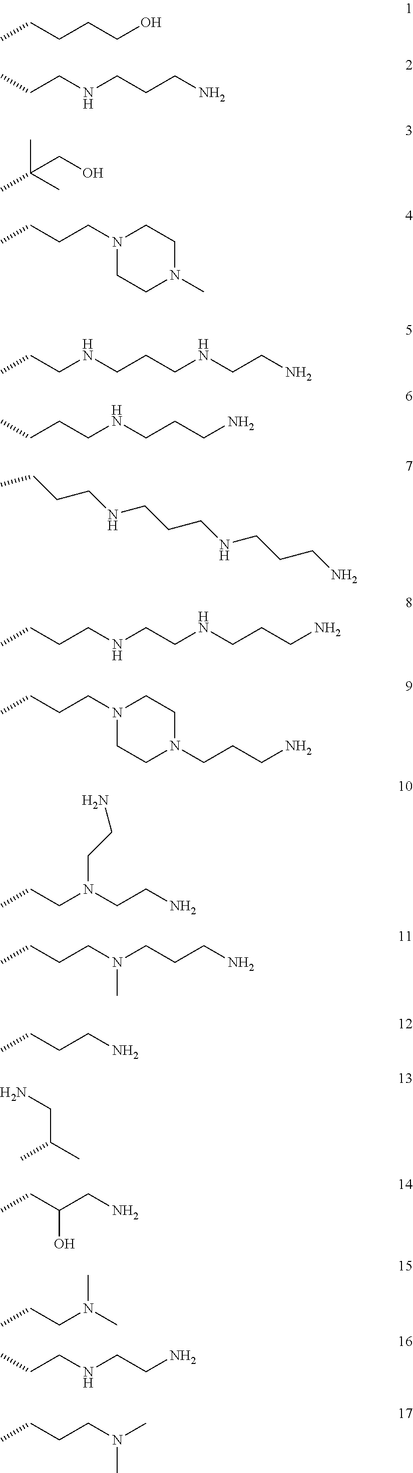

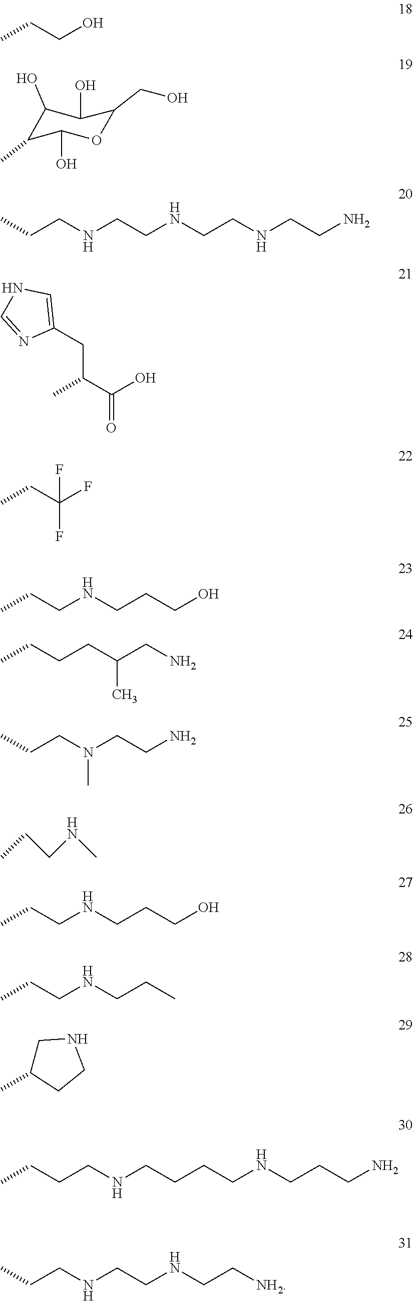

In some forms, the second PACEng polymer is as described above, except that the second PACEng polymer has a structure of Formula III.

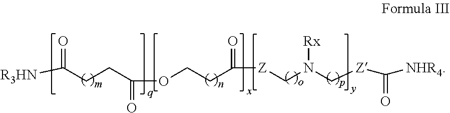

The monomer units can be substituted at one or more positions with one or more substituents. Exemplary substituents include, but are not limited to, alkyl groups, cyclic alkyl groups, alkene groups, cyclic alkene groups, alkynes, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The second PACE polymer (i.e., second PACEab polymer or second PACEng polymer) is preferably biocompatible. Readily available lactones of various ring sizes are known to possess low toxicity: for example, polyesters prepared from small lactones, such as poly(caprolactone) and poly(p-dioxanone) are commercially available biomaterials which have been used in clinical applications. Large (e.g., C₁₆-C₂₄) lactones and their polyester derivatives are natural products that have been identified in living organisms, such as bees. Lactones containing ring carbon atoms between 16 and 24 are specifically contemplated and disclosed.

In some forms, the second PACE polymers (i.e., second PACEab polymer or second PACEng polymer) can be further activated via temperature-controlled hydrolysis, thereby exposing one or more activated end group(s). The one or more activated end group(s) can be, for example, hydroxyl or carboxylic acid end groups, both of which can be generated via hydrolysis of ester bonds within the polymers. The activated second PACE polymers (i.e., second PACEab polymer or second PACEng polymer) can have a weight-average molecular weight between about 5 and 25 kDa, preferably between about 5 and 10 kDa.

In some forms, the nanoparticles of contain between about 1% wt/wt and about 15% wt/wt PACE-PAO polymer, and between about 99% wt/wt and about 15% wt/wt second PACE polymer (i.e., i.e., second PACEab polymer or second PACEng polymer), such as about 10% wt/wt PACE-PAO polymer and about 90% wt/wt second PACE polymer.

As used herein, the term “about” is meant to minor variations within acceptable parameters. For the sake of clarity, “about” refers to ±10% of a given value. In some forms, the activated second PACEng polymers contain R₁ or R₂ (such as chemical entities containing a hydroxyl group, a primary amine group, a secondary amine group, a tertiary amine group, or combinations thereof, exemplified by the non-limiting moieties denoted R₃ or R₄) at one end, and a hydroxyl or carboxylic acid end group at the other end, generated via hydrolysis.

In some forms, the second PACEng polymer is as described above, except that the second PACEng polymer has a structure of Formula IV.

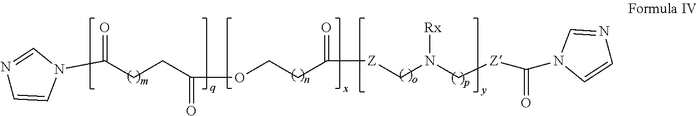

In some forms, the second PACEng polymer is as described above, except that the second PACEng polymer has a structure of Formula V.

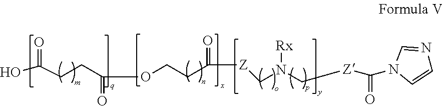

In some forms, the second PACEng polymer is as described above, except that the second PACEng polymer has a structure of Formula VI.

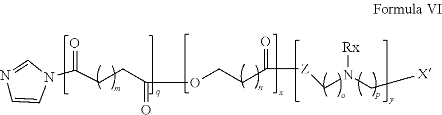

wherein X′ is —OH or —NHR′.

Formulas VI, V, and VI are structures of intermediary products. They can be used to synthesize a wide variety of second PACEng polymers with a structure of Formula I, II or III, wherein at least one of R₁ and R₂ is present.

3. Nanoparticles Containing Non-Covalently Conjugated Surfactant on the Surface, Optionally Bound to Targeting Moiety

In another aspect, the nanoparticles contain a surfactant non-covalently conjugated to the surface of the nanoparticles. In these forms, the nanoparticles contain a second PACE polymer, as described above under the section titled Nanoparticles containing PACE-PAO blended with a second PACE polymer. Preferably, the surfactant is a polymeric surfactant.

Preferably in these forms, the surface of the nanoparticles contains a surfactant to which a targeting moiety is covalently conjugated. The surfactant, containing the targeting moiety, contains a structure:

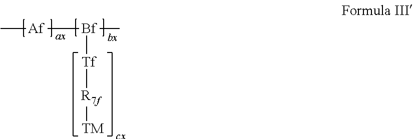

wherein:

each Af is independently a hydrophobic or hydrophilic monomer residue,

Bf is substituted alkyl, substituted aryl, substituted cycloalkyl, substituted heterocyclyl, substituted amine, substituted carbonyl, substituted amide, or substituted sulfone,

wherein Tf is independently oxygen or is absent,

wherein R_{7f} is independently hydrogen, alkyl, substituted alkyl, amide, substituted amide, substituted sulfone, unsubstituted sulfone, aryl, substituted alkyl, cycloalkyl, substituted cycloalkyl, maleimide, amine, substituted amine, thiol, N-hydroxysuccinimide ester, succinimide, azide, acrylate, methacrylate, alkyne, hydroxide, or isocynate,

TM is a targeting moiety,

ax is independently an integer between 0 and 10,000, between 0 and 5,000, or between 0 and 1,000, and

bx and cx are independently an integer between 1 and 10,000, between 1 and 5000, or between 1 and 1,000.

In some forms, the surfactant containing the targeting moiety is as described above, except that the surfactant contains a structure:

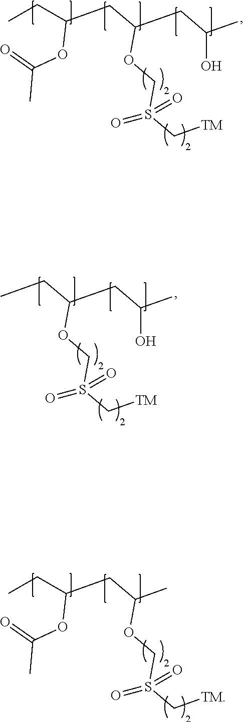

In either approach to conjugating targeting moieties to the nanoparticles, targeting moieties include small molecules (molecular weight between 200 Da and 2,500 Da), aptamers, proteins, or peptides.

In some forms, the nanoparticles include a cell-type or cell-state specific targeting domain or targeting signal. Examples of moieties which may be linked or unlinked to the nanoparticles include, for example, targeting moieties which provide for the delivery of molecules to specific cells. The targeting signal or sequence can be specific for a host, tissue, organ, cell, organelle, non-nuclear organelle, or cellular compartment. Moreover, the compositions here can be targeted to other specific intercellular regions, compartments, or cell types.

In some forms, the targeting signal binds to its ligand or receptor which is located on the surface of a target cell such as to bring the vector and cell membranes sufficiently close to each other to allow penetration of the vector into the cell. Additional embodiments are directed to specifically delivering polynucleotides to specific tissue or cell types, wherein the polynucleotides can encode a polypeptide or interfere with the expression of a different polynucleotide. The polynucleotides delivered to the cell can encode polypeptides that can enhance or contribute to the functioning of the cell.

The targeting moiety can be an antibody or antigen binding fragment thereof, an antibody domain, an antigen, a T-cell receptor, a cell surface receptor, a cell surface adhesion molecule, a major histocompatibility locus protein, a viral envelope protein and a peptide selected by phage display that binds specifically to a defined cell.

In some forms, preferably the targeting moiety is a protein or peptide. One skilled in the art will appreciate that the tropism of the nanoparticles described can be altered by merely changing the targeting signal. It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used as targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest.

B. Materials to be Bound to and/or Encapsulated in Nanoparticles

1. Therapeutic, Prophylactic and Diagnostic Agents

Active agents include synthetic and natural proteins (including enzymes, peptide-hormones, receptors, growth factors, antibodies, signaling molecules), and synthetic and natural nucleic acids (including RNA, DNA, anti-sense RNA, triplex DNA, inhibitory RNA (RNAi), and oligonucleotides), sugars and polysaccharides, small molecules (typically under 1000 Daltons), lipids and lipoproteins, and biologically active portions thereof. Suitable active agents have a size greater than about 1,000 Da for small peptides and polypeptides, more typically at least about 5,000 Da and often 10,000 Da or more for proteins. Nucleic acids are more typically listed in terms of base pairs or bases (collectively “bp”).

Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), danazole, and combinations thereof. Other suitable anti-cancer agents include angiogenesis inhibitors; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; and transforming growth factor-α or transforming growth factor-β inhibitors.

Other therapeutics that may be delivered with this technology include antibiotics (antibacterial, antiviral or antifungal), and antiinflammatory (such as steroids like cortisone and prednisone and non-steroidal antiinflammatories such as naproxen).

Imaging agents such as radioopaque compounds may also be incorporated to facilitate localization at the time of placement or removal.

Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents. Examples of other suitable contrast agents include gases or gas emitting compounds, which are radioopaque. Nanoparticles can further include agents useful for determining the location of administered particles. Agents useful for this purpose include fluorescent tags, radionuclides and contrast agents.

2. Loading

The percent loading is typically from about 1% to about 80%, from about 1% to about 50%, from about 1% to about 40% by weight, from about 1% to about 20% by weight, or from about 1% to about 10% by weight. In some embodiments, the percent drug loading is between about 5% and about 50%, or about 10% and about 40%, or about 15% and about 30%. In specific embodiments, drug loading is about 20, 21, 23, 24, 25, 26, 27, 28, 20, or 30%.

### III. Methods of Making and Reagents therefor

(i) Polymers

(a) Poly(amine-co-ester)s or poly(amine-co-amide)s with an Endgroup Modification or with poly(ethylene glycol)

The polymers are generally modified from synthetic polymers. Exemplary synthetic polymers include poly(amine-co-ester), formed of a lactone, a dialkyl acid, and a dialkyl amine Methods for the synthesis of poly(amine-co-ester) from a lactone, a dialkyl acid, and a dialkyl amine using an enzyme catalyst, such as a lipase, are also provided. Exemplary lactones are disclosed in U.S. Patent Publication No. US20170121454. In some forms, poly(amine-co-ester) is prepared as shown in Scheme 1:

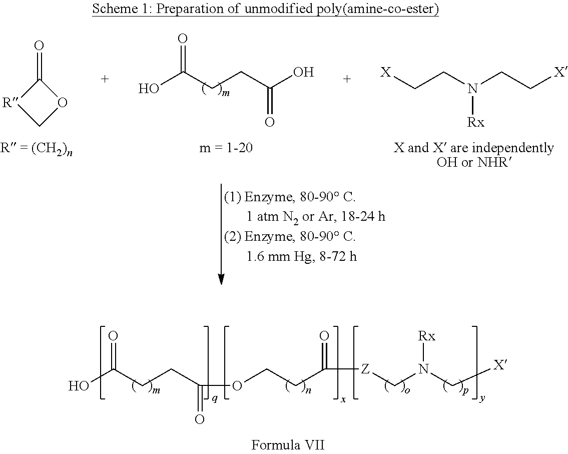

The synthesis of the polymers described herein using PDL, DES, MDA, and PEG as reactants is shown in Scheme 2.

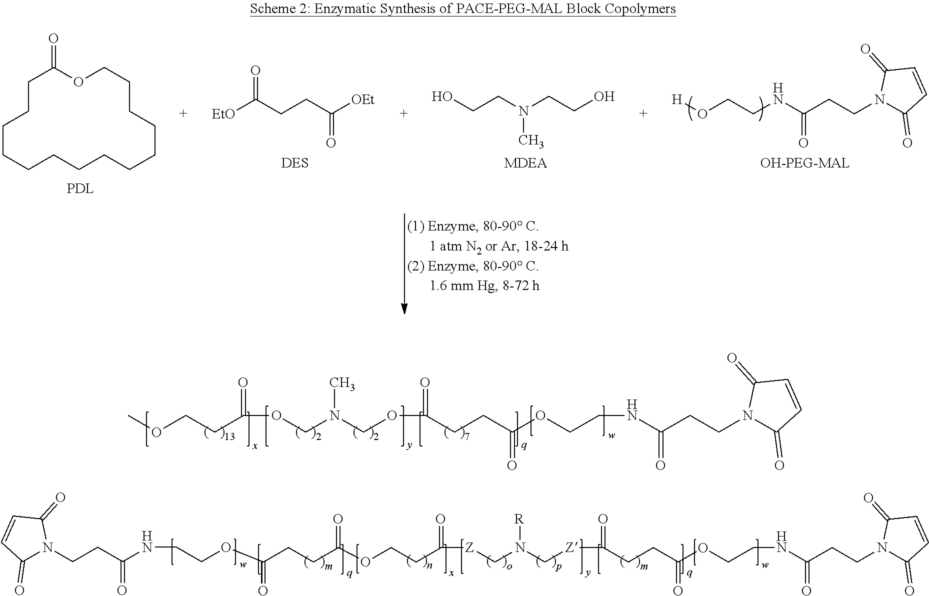

The molar ratio of the monomers (e.g., lactone:aminodiol:diacid) can vary, for example from about 10:90:90 to about 90:10:10. In some embodiments, the ratio is 10:90:90, 20:80:80, 40:60:60, 60:40:40, or 80:20:20. The weight average molecular weight, as determined by GPC using narrow polydispersity polystyrene standards, can vary, for example, from about 2,000 Daltons to about 50,000 Daltons, preferably from about 2,000 Daltons to about 20,000 Daltons, more preferably from about 5000 Daltons to about 20,000 Daltons, most preferably from about 5000 Daltons to about 10,000 Daltons.

The hydrophobicity of the polymers can be adjusted by varying the percentages of lactone, such as between about 10% and about 100% (calculated lactone unit vs. (lactone unit+diester/diacid)), or between about 30% and about 100%. The molecular weight of the polymers can be adjusted by tuning the second stage reaction time, such as between about 8 and about 72 h.

The enzymatic method allows for the synthesis of polymers with diverse chain structures and tunable hydrophobicities. In some embodiments, the hydrophobicity is varied by varying the ring size and/or molar amount of the lactone monomer. Lactone with a wide range of ring sizes (e.g., C₄-C₂₄, preferably C₆-C₂₄, more preferably from C₆-C₁₆) can be used as comonomers. The reaction can be performed in a single step without protection and deprotection of the amino group(s). Such amino-bearing copolyesters are extremely difficult to prepare using conventional organometallic catalysts, as such catalysts are often sensitive to or deactivated by organic amines. These catalysts are also known to be inefficient for polymerizing large lactone ring monomers. Enzymatic catalysts have distinct advantages for producing biomedical polymers owing to the high activity and selectivity of the enzyme and the resulting high purity of products that are metal-free.

Polymers with a structure of Formula IV, V, or VI can be synthesized via reacting the unmodified polymer of Formula VII with 1,1′-carbonyldiimidazole (CDI), at a molar ratio from about 1:10 to about 1:60, preferably at about 1:40.

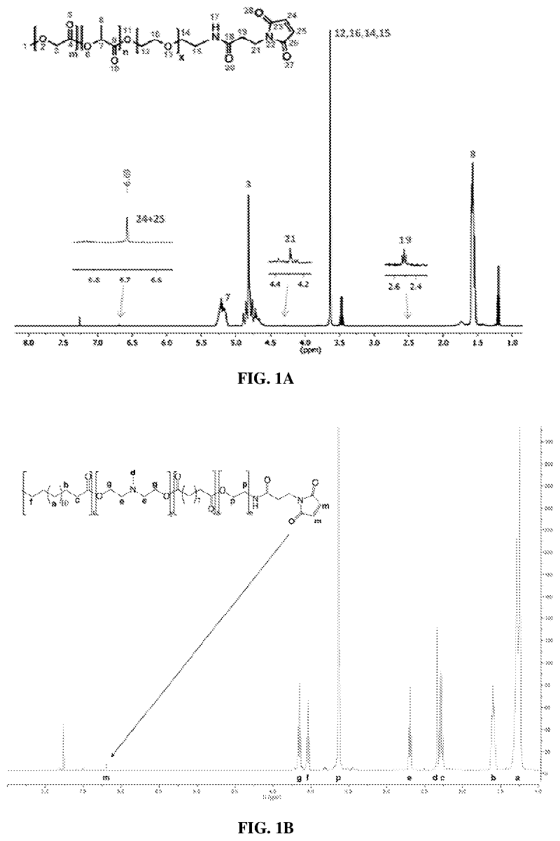

Polymers with a structure of Formulas I or II can be obtained via modification of the end groups of the unmodified polymer of Formula VII using coupling reactions known in the art. For example, polymers with a structure of Formula III can be synthesized via (1) reacting the unmodified polymer of Formula VII with CDI to obtain a polymer of Formula IV, and (2) reacting the polymer of Formula IV with R₃—NH₂ and R₄—NH₂. In some forms, R₃, R₄, or both are selected from those shown in FIG. 1. Preferably, R₃ and R₄ are the same.

Alternatively, polymers with a structure of Formula III can be synthesized via (1) reacting the unmodified polymer of Formula VII with CDI to obtain a polymer of Formula V or VI, (2) protecting the —COOH group or the —X′ group in the polymer from step (1), (3) reacting the protected polymer from step (2) with R₄—NH₂ or R₃—NH₂, (4) deprotecting the —COOH group or the —X′ group in the polymer from step (3), and (5) reacting the deprotected polymer from step (4) with R₃—NH₂ or R₄—NH₂.

Hydrolysis-mediated activation of the polymers of Formula I, II, or III can be performed in a temperature-controlled manner for up to 30 days or more. The length of hydrolysis may vary depending on the molecular weight of the polymers to be activated. Larger molecular weight polymers (e.g., about 20-25 kDa) are optimally hydrolyzed for longer periods of time, for example, for about 30 to 40 days. Smaller molecular weight polymers (e.g., about 5-7 kDa) are optimally hydrolyzed for shorter periods of time, for example, for about 4 to 10 days.

In some forms, the polymers are hydrolyzed at a temperature from about 30° C. to 42° C., or any in the range of up to about 100° C. The PACE polymers can be hydrolyzed at a temperature from about 35° C. to 40° C., e.g., about 37° C.

In some forms, the polymers are hydrolyzed, for example, at about 1 atm. Higher pressures accelerate the process (e.g., pressures from about 1 to about 100 atm). The rate for the process would be determined by one of skill in the art for the specific formulations being made.

The weight-average molecular weight of the resulting hydrolysis product can vary from about 5 kDa to about 25 kDa, preferably between about 5 and about 10 kDa.

Preferably, one or more of the ester bond in the polymers are hydrolyzed. The hydrolysis product can have R₁ or R₂ at one end and a carboxyl or a hydroxyl group at the other end, generated via hydrolysis.

(b) Synthesis of Poly(vinyl alcohol-vinyl sulfone)

PVA-VS polymer was prepared as described by Raudszus, et al. (Raudszus, et al., Int. J. Pharm. 2018, 536(1), 211-221). Further details are provided in the Examples.

(ii) Nanoparticles Particles can be prepared using a variety of techniques known in the art. The technique to be used can depend on a variety of factors including the polymer used to form the nanoparticles, the desired size range of the resulting particles, and suitability for the material to be encapsulated.

Methods known in the art that can be used to prepare nanoparticles include, but are not limited to, single and double emulsion; nanoprecipitation, nanoparticle molding; electrostatic self-assembly (e.g., polyethylene imine-DNA or liposomes); and polyelectrolyte condensation (see Suk et al., Biomaterials, 27, 5143-5150 (2006)). In preferred forms, the nanoparticles are prepared via single or double emulsion evaporation procedures.

In some forms, the loaded particles are prepared by combining a solution of the polymers, typically in an organic solvent, with an agent of interest (e.g., nucleic acid such as mRNA). First, a solution of the polymers is prepared by dissolving or suspending the polymer in a solvent(s) suitable to dissolve the polymers. The solvent(s) should be selected that does not adversely affect (e.g., destabilize or degrade) the nucleic acid to be encapsulated. Suitable solvents include, but are not limited to DMSO and methylene chloride, i.e., dichloromethane (DCM). The concentration of the polymer in the solvent(s) can be varied as needed.

Next, the polymer solution is mixed with the agent to be encapsulated, such as a polynucleotide. The agent can be dissolved in a solvent to form a solution before combining it with the polymer solution. In some embodiments, the agent is dissolved in a physiological buffer before combining it with the polymer solution. The ratio of polymer solution volume to agent solution volume can be 1:1. The combination of polymer and agent are typically incubated for a few minutes to form particles before using the solution for its desired purpose, such as transfection. In some forms, the nanoparticles are formed by containing the solution containing the polymers, with nonsolvent of the polymers or a component of a polymer(s) to produce the particle. A polymer/polynucleotide solution can be incubated for 2, 5, 10, or more than 10 minutes before using the solution for transfection. The incubation can be at room temperature.

(a) Fabrication of PACE-PEG-MAL Nanoparticles

In the non-limiting case where the nanoparticles contain PACE-PEG-MAL, a targeting moiety can be conjugated to the polymer before or after formation of the nanoparticle. In a non-limiting example, where the targeting moiety is a protein or peptide, conjugation proceeds via a facile “click” reaction between a reactive group on the protein or peptide (e.g., a thiol) and the maleimide group on the polymer to form a succinimide group linking the targeting moiety to the PEG terminal of the polymer. The thiol can be from the side chain of a cysteine residue in the protein or peptide.

(b) Fabrication of PACE-PVA-VS

In the non-limiting case where the nanoparticles contain PACE-PVA-VS, a targeting moiety can be conjugated to the PVA-VS polymer before or after formation of the nanoparticle. In a non-limiting example, where the targeting moiety is a protein or peptide, conjugation proceeds via a facile “click” reaction between a reactive group on the protein or peptide (e.g., a thiol) and the free vinyl group of the vinyl sulfone covalently conjugated to the PACE-VS polymer. The thiol can be from the side chain of a cysteine residue in the protein or peptide. In a non-limiting example, the PACE polymer is dissolved in an organic phase and then added to a PVA-VS solution to form an emulsion. The resulting emulsion can next be transferred to a second solution of PVA followed by evaporation of the organic phase to form nanoparticles.

### IV. Methods of Using

The particles can be used to deliver an effective amount of one or more therapeutic, diagnostic, and/or prophylactic agents to a patient in need of such treatment. The amount of agent to be administered can be readily determine by the prescribing physician and is dependent on the age and weight of the patient and the disease or disorder to be treated.

In some forms, the agents include nucleic acids, such as mRNA. In some forms, the agents are non-covalently encapsulated within the nanoparticles. In some forms, the nanoparticles have a higher proportion of agents encapsulated with the nanoparticles than on the surface of the nanoparticles. In some forms, the agents are encapsulated within the nanoparticles and are not on the surface of the nanoparticles.

The nanoparticles have several advantages over other delivery systems. The nanoparticles can have improved loading of nucleic acids, and can also display controlled orientation of targeting agents (such as proteins and peptides) on the surfaces of the nanoparticles. In a non-limiting example, the orientation of targeting agents (such as proteins and peptides) can be achieved by covalent conjugation via a specific amino acid (e.g., involving the thiol group of a cysteine) present in the proteins or peptides. These properties make it possible to deliver agents, such as nucleic acids to targeted tissues without induction of undesirable side effects, such as systemic toxicity. Therefore, the nanoparticles serve as a versatile platform for delivery (e.g., controlled delivery) of nucleic acids.

The particles can be administered intravenously, subcutaneously, or intramuscularly, administered to the nasal or pulmonary system, injected into a tumor milieu, administered to a mucosal surface (vaginal, rectal, buccal, sublingual), or encapsulated for oral delivery. The particles may be administered as a dry powder, as an aqueous suspension (in water, saline, buffered saline, etc), in a hydrogel, organogel, or liposome, in capsules, tablets, troches, or other standard pharmaceutical excipient.

(i) Vaccine Platform

The nanoparticles can be used as a vaccine delivery platform, wherein they include antigens, or nucleic acids encoding antigens to provide enhanced immunity against pathogens. In a non-limiting example, the nanoparticles can be used in methods for inducing or enhancing a robust mucosal immunity to an exogenous antigen in mucosal and epithelial tissues of a subject are provided. The methods effectively generate one or more populations of tissue resident T cells and B cells in the recipient. In some forms, the methods provide a population of tissue resident memory T and B cells protective against the vaccinating antigen(s) and/or the original host where the vaccinating antigen is sourced. In some forms the methods induce an antibody response in mucosal or epithelial tissue. Preferably, the antibody response is composed primarily of IgA antibodies. A cellular mucosal immune response may be assessed by measuring the T cell response from lymphocytes isolated from the mucosal area (e.g., respiratory, or gastrointestinal tract) or from lymph nodes that drain from the mucosal area (for example respiratory area or gastrointestinal area). In some forms, the methods provide protective immunity against infectious diseases. In particular, the methods provide protective immunity against one or more respiratory diseases. The diseases for which protective immunity is provided are determined by the antigen that is provided within the compositions for administration.

In some forms, the methods vaccinate a subject against infection with a respiratory viral disease, such as infection with a coronavirus associated with the development of severe acute respiratory syndrome (SARS), or an influenza virus.

(ii) Transfection Platform

The nanoparticles can be for cell transfection of polynucleotides. The transfection can be applied in applications including gene therapy and disease treatment. The nanoparticles can be more efficient, less toxic, or a combination thereof when compared to a control. In some embodiments, the control is cells treated with an alternative transfection reagent such as LIPOFECTAMINE 2000 or polyethylenimine (PEI).

Transfection is carried out by contacting cells with the solution containing the nanoparticles. For in vivo methods, the contacting typically occurs in vivo after the solution is administered to the subject. For in vitro methods, the solution is typically added to a culture of cells and allowed to contact the cells for minutes, hours, or days. The cells can subsequently be washed to move excess nanoparticles.

The particular polynucleotide delivered by the nanoparticles can be selected by one of skill in the art depending on the condition or disease to be treated. The polynucleotide can be, for example, a gene or cDNA of interest, mRNA, a functional nucleic acid such as an inhibitory RNA, a tRNA, an rRNA, or an expression vector encoding a gene or cDNA of interest, a functional nucleic acid a tRNA, or an rRNA. In some forms two or more polynucleotides can be administered in combination.

The nanoparticles can be used in a method of delivering polynucleotides to cells in vitro. For example, the nanoparticles can be used for in vitro transfection of cells. The method typically involves contacting the cells with nanoparticles including a polynucleotide in an effective amount to introduce the polynucleotide into the cell's cytoplasm. In some forms, the polynucleotide is delivered to the cell in an effective amount to change the genotype or a phenotype of the cell. The cells can primary cells isolated from a subject, or cells of an established cell line. The cells can be of a homogenous cell type, or can be a heterogeneous mixture of different cell types. For example, the nanoparticles can be introduced into the cytoplasm of cells from a heterogenous cell line possessing cells of different types, such as in a feeder cell culture, or a mixed culture in various states of differentiation. The cells can be a transformed cell line that can be maintained indefinitely in cell culture. Exemplary cell lines are those available from American Type Culture Collection including tumor cell lines.

Any eukaryotic cell can be transfected to produce cells that express a specific nucleic acid, for example a metabolic gene, including primary cells as well as established cell lines. Suitable types of cells include, but are not limited to, undifferentiated or partially differentiated cells including stem cells, totipotent cells, pluripotent cells, embryonic stem cells, inner mass cells, adult stem cells, bone marrow cells, cells from umbilical cord blood, and cells derived from ectoderm, mesoderm, or endoderm. Suitable differentiated cells include somatic cells, neuronal cells, skeletal muscle, smooth muscle, pancreatic cells, liver cells, and cardiac cells. In some forms, siRNA, antisense polynucleotides (including siRNA or antisense polynucleotides) or inhibitory RNA can be transfected into a cell using the nanoparticles.

The methods are also useful in the field of personalized therapy, for example, to repair a defective gene, de-differentiate cells, or reprogram cells. For example, target cells are first isolated from a donor using methods known in the art, contacted with the nanoparticles including a polynucleotide causing a change to the in vitro (ex vivo), and administered to a patient in need thereof. Sources or cells include cells harvested directly from the patient or an allographic donor. In some forms, the target cells to be administered to a subject will be autologous, e.g., derived from the subject, or syngeneic. Allogeneic cells can also be isolated from antigenically matched, genetically unrelated donors (identified through a national registry), or by using target cells obtained or derived from a genetically related sibling or parent.

Cells can be selected by positive and/or negative selection techniques. For example, antibodies binding a particular cell surface protein may be conjugated to magnetic beads and immunogenic procedures utilized to recover the desired cell type. It may be desirable to enrich the target cells prior to transient transfection. As used herein in the context of compositions enriched for a particular target cell, “enriched” indicates a proportion of a desirable element (e.g. the target cell) which is higher than that found in the natural source of the cells. A composition of cells may be enriched over a natural source of the cells by at least one order of magnitude, preferably two or three orders, and more preferably 10, 100, 200, or 1000 orders of magnitude. Once target cells have been isolated, they may be propagated by growing in suitable medium according to established methods known in the art. Established cell lines may also be useful in for the methods. The cells can be stored frozen before transfection, if necessary.

Next the cells are contacted with the composition in vitro to repair, de-differentiate, re-differentiate, and/or re-program the cell. The cells can be monitored, and the desired cell type can be selected for therapeutic administration.

Following repair, de-differentiation, and/or re-differentiation and/or reprogramming, the cells are administered to a patient in need thereof. In the most preferred embodiments, the cells are isolated from and administered back to the same patient. In alternative embodiments, the cells are isolated from one patient, and administered to a second patient. The method can also be used to produce frozen stocks of altered cells which can be stored long-term, for later use. In some forms, fibroblasts, keratinocytes or hematopoietic stem cells are isolated from a patient and repaired, de-differentiated, or reprogrammed in vitro to provide therapeutic cells for the patient.

In some in vivo approaches, the nanoparticles are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

Pharmaceutical compositions can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some forms, the nanoparticles are administered systemically, for example, by intravenous or intraperitoneal administration, in an amount effective for delivery of the compositions to targeted cells. Other possible routes include trans-dermal or oral.

In certain forms, the nanoparticles are administered locally, for example by injection directly into a site to be treated. In some forms, the compositions are injected or otherwise administered directly to one or more tumors. Typically, local injection causes an increased localized concentration of the nanoparticles which is greater than that which can be achieved by systemic administration. In some forms, the nanoparticles are delivered locally to the appropriate cells by using a catheter or syringe. Other means of delivering such nanoparticles locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the nanoparticles to the immediate area of the implant.

The nanoparticles can be provided to the cell either directly, such as by contacting it with the cell, or indirectly, such as through the action of any biological process. For example, the nanoparticles can be formulated in a physiologically acceptable carrier or vehicle, and injected into a tissue or fluid surrounding the cell. The nanoparticles can cross the cell membrane by simple diffusion, endocytosis, or by any active or passive transport mechanism.

The nanoparticles can be used in gene therapy protocols for the treatment of gene related diseases or disorders. Cell dysfunction can also be treated or reduced using the compositions and methods. In some forms, diseases amenable to gene therapy are specifically targeted. The disease can be in children, for example individuals less than 18 years of age, typically less than 12 years of age, or adults, for example individuals 18 years of age or more. Thus, embodiments of the present disclosure are directed to treating a host diagnosed with a disease, by transfection of the nanoparticles including a polynucleotide into the cell affected by the disease and wherein the polynucleotide encodes a therapeutic protein. In some forms, an inhibitory RNA is directed to a specific cell type or state to reduce or eliminate the expression of a protein, thereby achieving a therapeutic effect. The present disclosure encompasses manipulating, augmenting or replacing genes to treat diseases caused by genetic defects or abnormalities.

The present invention will be further understood by reference to the following non-limiting examples.

### EXAMPLES

### Example 1: Targeted PACE NPs Effectively Direct the Nanoparticles to Target Cells In Vitro

Linear poly(amine-co-ester) terpolymers can form efficient delivery systems for nucleic acids, including mRNA. Here, two different methods for attachment of peptides and proteins to the surface of nanoparticles formed from PACE were designed and assessed for enhanced targeting of these nanoparticles. These methods allow for the efficient and versatile targeting of these nanoparticles to cells or other components of tissues.

### Materials and Methods

(i) Conjugation Method 1

(a) Synthesis of PACE-PEG-Maleimide

PACE-PEG-Maleimide was synthesized via copolymerization of 15-pentadecanolide, N-methyl diethanolamine, diethyl sebacate, and Maleimide-PEG-OH in diphenyl ether using a lipase-based enzyme catalyst (CALB). In the first stage (oligomerization) the reactants and solvent were stirred at 250 rpm at 90° C. under 1 atm of argon gas for 18-20 hours. In the second stage (polymerization), the reaction was continued for an additional 48-72 hours under vacuum. The resulting crude polymer was then washed in hexane three times, dissolved in chloroform, and filtered. The added chloroform was then removed via a rotary evaporator.

(b) Fabrication of DiD-Loaded PACE-PEG-MAL Nanoparticles by Single Emulsion Evaporation

DiD-loaded PACE-PEG-MAL nanoparticles were synthesized following standard single emulsion evaporation procedures.

(c) Conjugation of Cysteine-Terminated Binding Protein to PACE-PEG-MAL on Nanoparticles and Cellular Uptake of Dye-Loaded Nanoparticles

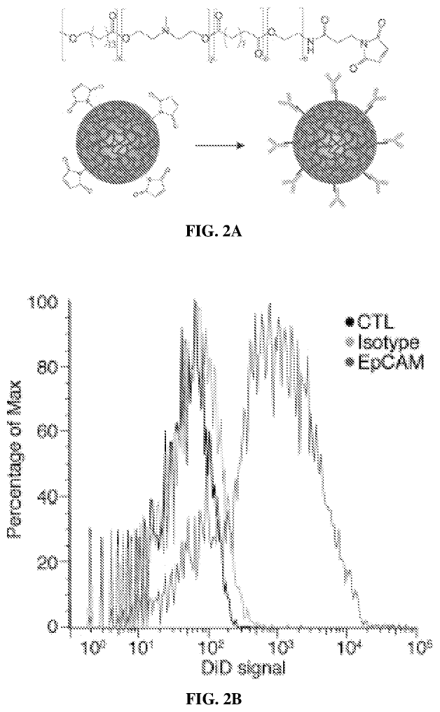

PACE-PEG-MAL NPs were conjugated with a Cys-terminated protein using the thiol reaction between the maleimide groups on the surface of the NPs and the thiol group single terminal cysteine of the protein, FIG. 2A. The reaction was carried out at room temperature with mild mixing (Orbit Shaker, 250 rpm) for 1 h with a NPs:protein weight ratio of 10:1. Excess protein was removed by centrifugation (15 min, 25 000 rpm, 17° C.) and the NPs redispersed in PBS at a concentration of 5 mg/mL. Binding protein-NPs were flash frozen in liquid nitrogen and stored at −80° C. until use.

Human bronchial epithelial cells homozygous for the W128X nonsense cystic fibrosis mutation were treated with dye (DiD)-loaded PACE-PEG-MAL nanoparticles functionalized with antibodies targeting the epithelial marker EpCAM or isotype control antibodies. Cellular uptake was measured by flow cytometry and fluorescence microscopy.

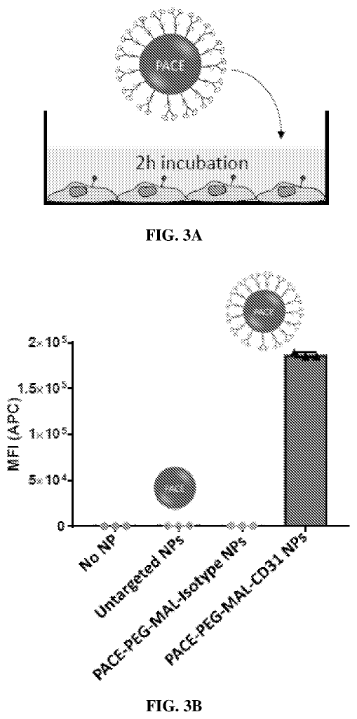

Human umbilical vein endothelial cells were also used to assess uptake of targeted, untargeted, and control nanoparticles. FIG. 3A.

(ii) Conjugation Method 2

(a) Synthesis of Poly(Vinyl Alcohol-Vinyl Sulfone) (PVA-VS)

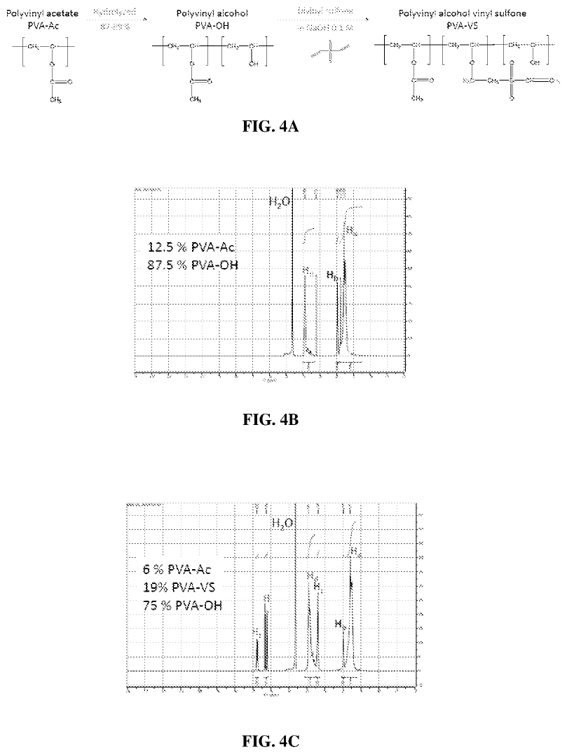

PVA-VS polymer was prepared as described by Raudszus, et al. (Raudszus, et al., Int. J. Pharm. 2018, 536(1), 211-221). The excess of divinyl sulfone was removed by dialysis for 24 h against water (Slide-ALyzer ®Dialysis Cassette, 10 000 MCWO, Thermo Scientific). The resulting PVA-VS was lyophilized and characterized by 1H NMR in D20 (Agilent DD2 400 MHz). A schematic for the synthesis of PVA-VS is shown in FIG. 4A.

(b) Fabrication of PACE-PVA-VS by Single Emulsion Evaporation

PACE-PVA-VS NPs were prepared following an emulsion-evaporation method. The polymer was dissolved in DCM at 50 mg/mL and mixed with lipophilic fluorescent dye (DiD) at a dye:polymer weight ratio of 0.5%. This organic phase was then added dropwise with a glass pasteur pipette into a PVA-VS solution at 5% (w/w) under vigorous vortexing (organic:aqueous phase volume ratio of 1:2). The resulting emulsion was then transferred into a beaker containing three times its volume of PVA solution at 0.3% (w/w) under agitation. After 1 min the organic solvent was evaporated using a rotary evaporator. Excess of PVA and PVA-VS were washed by two centrifugations (18 000 g, 45 min, 4° C.). The pellet was redispersed in DI water, flash frozen in liquid nitrogen and stored at −80° C. The size and potential zeta of the NPs were measured by dynamic light scattering (Zetasizer Nano ZS, He—Ne laser 633 nm, 173°, Smoluchowski equation, Malvern Panalytical).

(c) Surface Attachment of Protein to PACE-PVA-VS Nanoparticles Through the Use of PVA-VS and Cellular Uptake of Dye-Loaded Nanoparticles

In a first step, PACE-PVA-VS NPs were conjugated with the Cys-terminated protein using the thiol reaction between the vinyl sulfone groups on the surface of the NPs and the thiol group of single terminal cysteine of the protein. The reaction was carried out at room temperature with mild mixing (Orbit Shaker, 250 rpm) for 1 h with a NPs:protein weight ratio of 10:1. Excess protein was removed by centrifugation (15 min, 25 000 rpm, 17° C.) and the NPs redispersed in PBS at a concentration of 5 mg/mL.

Human umbilical vein endothelial cells were used to assess uptake of targeted, control nanoparticles, and no nanoparticles. The cell nuclei were stained with DAPI.

### Results

(i) Conjugation Method 1

(a) Synthesis of PACE-PEG-Maleimide

The structure of the PACE-PEG-MAL polymer was confirmed via NMR, as shown in FIGS. 1A and 1B.

(b) Fabrication of DiD-Loaded PACE-PEG-MAL nanoparticles by single emulsion evaporation

The PACE-PEG polymer compositions and nanoparticle properties are shown in Table 1.

TABLE 1
PACE-PEG polymer compositions and nanoparticle properties

| | Z-average | | Zeta Potential |
|---|---|---|---|
| Polymer (all 60% PDL)^{a} | (nm) | PDI | (mV) |
| PACE-PEG-MAL (Batch 1) | 214 | 0.31 | 21.4 ± 4.3 |
| PACE-PEG-MAL (Batch 2) | 222 | 0.32 | 17.5 ± 3.7 |
| 50:50 PACE-PEG-MAL:PACE- | 175 | 0.19 | 23.1 ± 6.9 |
| PEG | | | |
| PACE-PEG | 170 | 0.22 | 28.4 ± 3.6 |

^{a}% is mole percent;
PDL = ω-pentadecalactone.

(c) Conjugation of Cysteine-Terminated Binding Protein to PACE-PEG-MAL on Nanoparticles and Cellular Uptake of Dye-Loaded Nanoparticles

FIG. 2A shows a schematic for the nanoparticle surface functionalization, taking advantage of the versatile maleimide chemistry. The EpCAM-targeting nanoparticles exhibited enhanced uptake compared to the isotype controls, as shown in FIG. 2B.

As shown in FIG. 3B, chemical conjugation of protein via PACE-PEG-MAL to form targeted nanoparticles (CD31 functionalized), increased targeted uptake in HUVECs compared to untargeted nanoparticles, and control nanoparticles (functionalized with CD31 isotype).

(ii) Conjugation Method 2

(a) Synthesis of PVA-VS

The structure of the PVA-VS polymer was confirmed via NMR, as shown in FIGS. 4B and 4C.

(b) Fabrication of PACE-PVA-VS by Single Emulsion Evaporation

The PACE-PVA-VS polymer compositions and nanoparticle properties are shown in Table 2.

TABLE 2
PACE-PVA-VS polymer compositions and nanoparticle properties

| | Z-average | | Zeta Potential |
|---|---|---|---|
| Polymer (all 60% PDL)^{a} | (nm) | PDI | (mV) |
| PACE-PVA-VS (Batch 1 - DiD | 310 | 0.15 | 11.0 ± 5.1 |
| loaded) | | | |
| PACE-PVA-VS (Batch 2 - DiD | 245 | 0.13 | 17.3 ± 5.7 |
| loaded) | | | |
| PACE-PVA-VS (FITC-dsDNA | 336 | 0.19 | 5.8 ± 3.8 |
| loaded) | | | |
| PACE-PVA (FITC-dsDNA | 255 | 0.14 | 16.0 ± 4.1 |
| loaded) | | | |
| PACE-PVA-VS (p65 siRNA | 226 | 0.10 | 15.1 ± 5.1 |
| loaded) | | | |

^{a}% is mole percent;
PDL = ω-pentadecalactone.

(c) Conjugation of Cysteine-Terminated Binding Protein to PACE-PVA-VS on Nanoparticles and Cellular Uptake of Dye-Loaded Nanoparticles

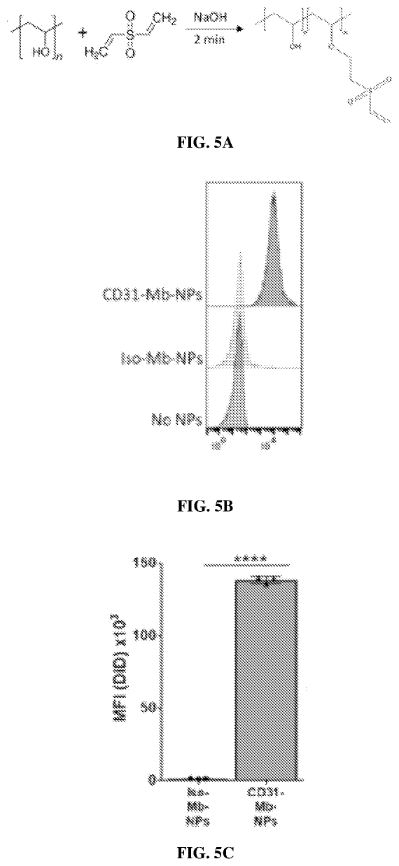

As shown in FIGS. 5B and 5C, chemical conjugation of protein to PACE nanoparticles via PVA-VS increased uptake of targeted nanoparticles (functionalized with CD31) in HUVECs compared to control nanoparticles (functionalized with CD31 isotype).

In summary, the results show that PACE-PEG-maleimide can be formulated into PACE nanoparticles to allow for efficient targeting of the nanoparticles to cells and tissues. Further, a surfactant, such as poly(vinyl alcohol-vinyl sulfone) can be added to the PACE nanoparticle surface during particle formation, and then used to conjugate a targeting protein for efficient targeting of the nanoparticles to cells and tissues.

### Example 2: In Vivo Uptake of Surface Modified Nanoparticles

### Materials and Methods

Blood samples from consenting healthy donors were collected at the Yale School of Medicine. All collections were performed under protocols approved by the Yale University Institutional Review Board (IRB). Participants were not compensated for their enrollment.

As described in Example 1, NPs were formulated using a 50:50 blend of PACE-PEG polymer and PACE-PEG-maleimide polymer encapsulating DiD dye. These NPs were conjugated first to a monobody (Mb) adapter (clone FCM101) with the thiol reaction between the maleimide groups on the surface of the NPs and the thiol group of single terminal cysteine of the Mb. The NPs and the Mbs are at a weight ratio of 10 NPs:1 Mb. This reaction was carried out at room temperature for 1 hour while shaking at 250 rpm. The excess Mb was removed by centrifugation (15 min, 18,000×g, 17° C.).

The NPs were redispersed in PBS at a concentration of 5 mg/mL, and then coupled with antibodies against human CD4 (clone 9H5A8) or rat CD4 (clone W3/25) at a weight ratio of 18 NPs:1Ab at room temperature for 1 hr with gentle mixing. Excess Ab was removed with centrifugation (15 min, 18,000×g, 17° C.). The resulting NPs were flash frozen and stored at −80° C. until use. Successful conjugation of the antibody was verified using a cell-based binding assay.

Dye-loaded NPs composed of 50:50 PACE-PEG:PACE-PEG-MAL formulated with anti-human-CD4 antibodies conjugated to the surface were incubated with isotype control NPs with whole human blood for 5 min, 12 min, 30 min, or 1 hour, before isolating PBMCs and assessing NP uptake in various cell types by flow cytometry.

In vivo targeting potential of PACE-PEG-MAL-based NPs was then assessed in wild type rats with decoy pre-administration of either isotype control or rat CD4-targeting NPs. The NPs were administered to wildtype rats 24 hours after clodronate liposome administration. Peripheral blood mononuclear cells (PBMCs) were isolated from peripheral blood samples 2 hours later and analyzed for NP uptake by flow cytometry.

### Results

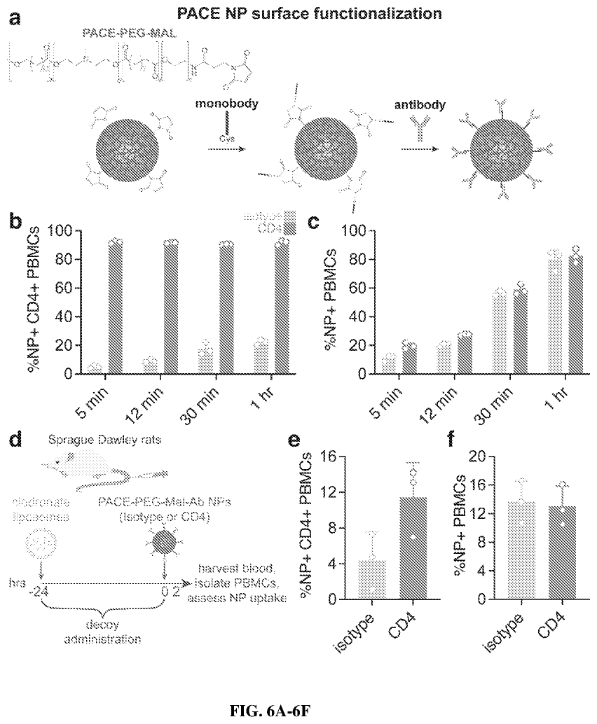

Results are shown in FIG. 6A-6F, showing that CD4-targeting PACE NPs accumulate selectively in CD4⁺ T-cells in vitro in human blood and in vivo in rats.

FIG. 6A shows surface functionalization of the nanoparticles formed of PACE-PEG-MAL using a monobody conjugated via the cysteine to the NP surface, then binding of antibody to the monobody, to form NP surface functionalized with CD4+.

The NPs were then added to PBMCs. Binding at 5, 12, 30 and 60 minutes is shown in FIG. 6B for isotype and CD4; binding of control NPs not surface functionalized is shown in FIG. 6C for isotype and CD4.

FIG. 6D shows the procedure where rats are treated with clodronate liposomes at −24 hours, then either isotype or CD4 bound PACE-PEG-Mal-Ab NPs injected into the tail vein of the rats. FIG. 6E shows the percentage of CD4+ NPs bound to PBMCs for isotype and CD4; FIG. 6F shows percentage of the control NPs not surface modified with CD4 for isotype and CD4.

The results demonstrate functionalization and efficacy of binding with the CD4 functionalized PACE-PEG-MAL NPs.

Those skilled in the art will recognize, or be able to ascertain using no more than routine experimentation, many equivalents to the specific embodiments of the invention described herein. Such equivalents are intended to be encompassed by the following claims.

We claim:

1. Nanoparticles comprising a poly(amine-co-ester)- or a poly(amine-co-amide) (PACE)-polyalkylene oxide (PACE-PAO) polymer having a structure:
((A)ₓ-(B)_{y}-(C)_{q}-(D)_{w}-(E)_{f})ₕ, Formula Ia′
wherein,
for Formula Ia′, A, B, C, D, and E independently include a monomeric lactone, a polyfunctional molecule, a diacid or diester, or polyalkylene oxide, wherein A, B, C, D, and E include at least a lactone, a polyfunctional molecule, a diacid or diester monomeric units, and polyalkylene oxide, x, y, q, w, and f are independently integers from 0-1000, with the proviso that the sum (x+y+q+w+f) is greater than one, and h is an integer from 1 to 1000.

2. The nanoparticles of claim 1, wherein the PACE-PAO comprises between about 0.1% wt/wt and about 95% wt/wt, between about 0.1% wt/wt and about 50% wt/wt, between about 0.1% wt/wt and about 20% wt/wt, between about 0.1% wt/wt and about 10% wt/wt, between about 0.1% wt/wt and about 5% wt/wt, between about 1% wt/wt and about 5% wt/wt, between 5% wt/wt and 10% wt/wt, or between about 10% wt/wt and about 20% wt/wt of the nanoparticles.

3. The nanoparticles of claim 1, wherein the polyalkylene oxide is a poly(ethylene glycol).

4. The nanoparticles of claim 1, wherein Formula Ia′ has a structure: 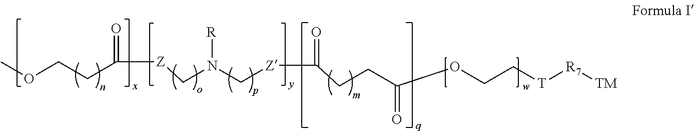
wherein:
for Formula I′, n is an integer from 1-30, m, o, and p are independently an integer from 1-20, x, y, and q, are independently integers from 1-1000, w is independently an integer from 0-1000, with the proviso that in Formula I′, w is greater than 0,
for Formula I′, Z and Z′ are independently O or NR′, wherein R and R′ are independently hydrogen, substituted or unsubstituted alkyl, or substituted or unsubstituted aryl,
for Formula I′, T is independently absent, oxygen, sulfur, alkyl, substituted alkyl, amide, substituted amide, amine, substituted amine, carbonyl, substituted carbonyl,
for Formula I′, R₇ is independently hydrogen, alkyl, substituted alkyl, amide, substituted amide, substituted sulfone, unsubstituted sulfone, aryl, substituted aryl, cycloalkyl, substituted cycloalkyl, maleimide, amine, substituted amine, thiol, N-hydroxysuccinimide ester, succinimide, azide, acrylate, methacrylate, alkyne, hydroxide, or isocynate, and
for Formula I′, TM is independently absent or a targeting moiety.

5. The nanoparticles of claim 4, wherein the targeting moiety is a protein or peptide.

6. The nanoparticles of claim 4, wherein the targeting moiety is selected from an antibody or antigen binding fragment thereof, an antibody domain, a T-cell receptor, a cell surface receptor, a cell surface adhesion molecule, a major histocompatibility locus protein, a viral envelope protein, a peptide selected by phage display that binds specifically to a defined cell, or a combination thereof.

7. The nanoparticles of claim 4, wherein the PACE-PAO polymer has a weight average molecular weight between 5,000 Daltons and 50 kDa, or between 5,000 Daltons and 30 kDa, wherein the weight average molecular weight does not include the molecular weight of the targeting moiety.

8. The nanoparticles of claim 1, comprising a mixture of the PACE-PAO polymer, and a second PACE polymer.

9. The nanoparticles of claim 8, wherein the second PACE polymer has a structure: 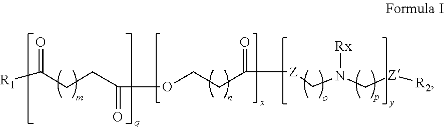
wherein,
for Formula I, n is an integer from 1-30, m, o, and p are independently integers from 1-20, x, y, and q are independently integers from 1-1000, Rₓ is hydrogen, substituted or unsubstituted alkyl, or substituted or unsubstituted aryl, or substituted or unsubstituted alkoxy, Z and Z′ are independently O or NR′, wherein R′ is hydrogen, substituted or unsubstituted alkyl, or substituted or unsubstituted aryl, R₁ and R₂ are independently absent or are chemical entities containing a hydroxyl group, a primary amine group, a secondary amine group, a tertiary amine group, or combinations thereof.

10. The nanoparticles of claim 9, wherein for Formula I, R₁ and/or R₂ are not

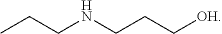

11. The nanoparticles of claim 9, wherein Formula I has a structure: 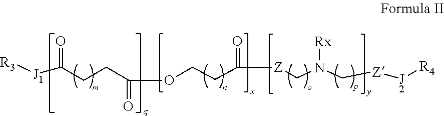
wherein J₁ and J₂ are independently linking moieties or absent,
R₃ and R₄ are independently substituted alkyl containing a hydroxyl group, a primary amine group, a secondary amine group, a tertiary amine group, or combinations thereof.

12. The nanoparticles of claim 9, wherein Formula I has a structure:

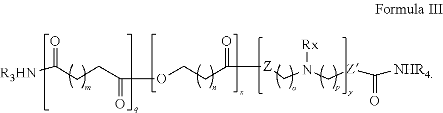

13. The nanoparticles of claim 9, wherein for Formula I n is 4, 10, 13, or 14.

14. The nanoparticles of claim 9, wherein for Formula I R_{X} is substituted or unsubstituted alkyl.

15. The nanoparticles of claim 9, wherein the second PACE polymer has a weight average molecular weight between about 2,000 Daltons and about 30,000 Daltons, between about 2,000 Daltons and about 25,000 Daltons, between about 2,000 Daltons and about 20,000 Daltons, or between about 5,000 Daltons and about 10,000 Daltons.

16. The nanoparticles of claim 11, wherein R₃ and/or R₄ are independently selected from the group consisting of

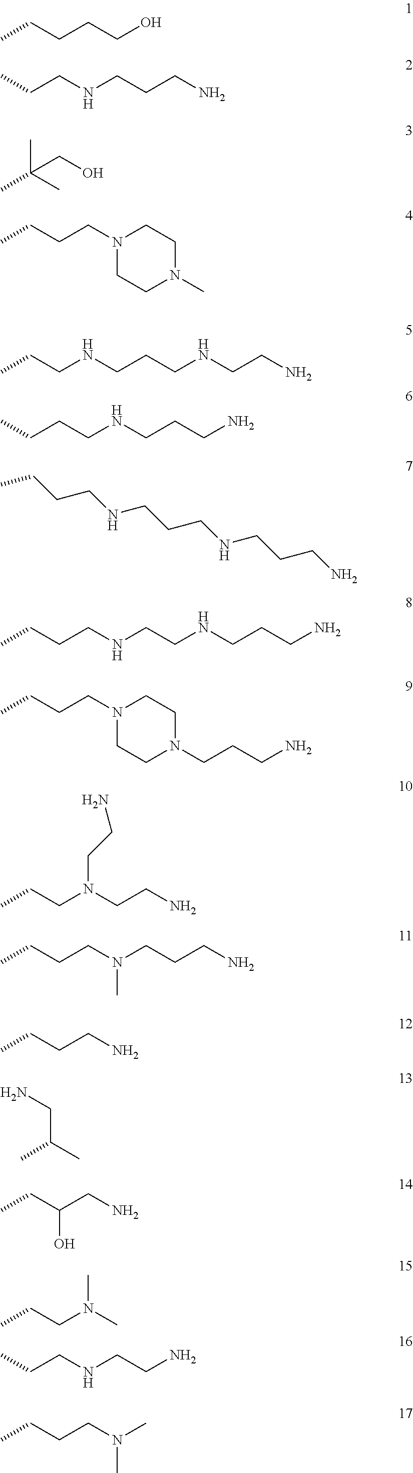

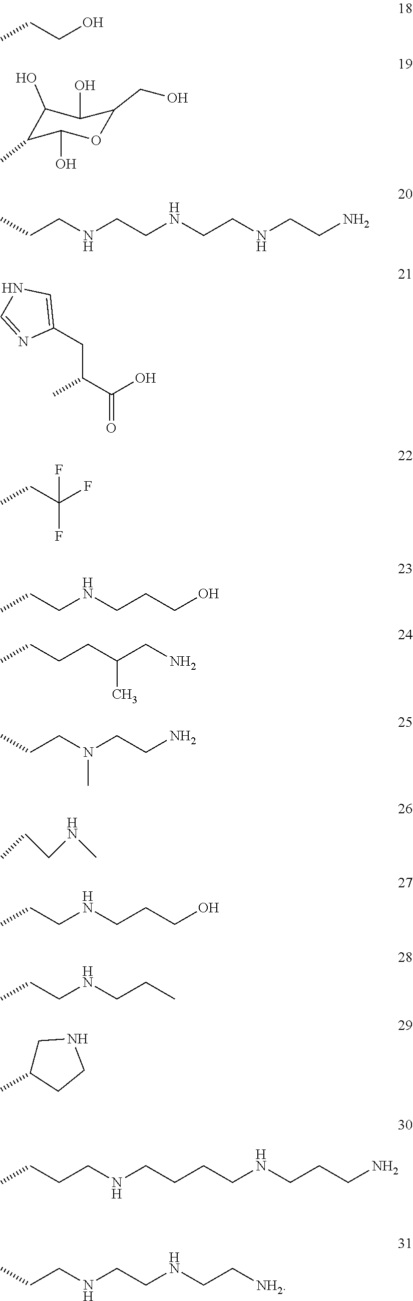

17. The nanoparticles of claim 8, comprising between about 1% wt/wt and about 15% wt/wt PACE-PAO polymer, and between about 99% wt/wt and about 15% wt/wt second PACE polymer.

18. Nanoparticles comprising:
(i) a core comprising a polymer having a structure: 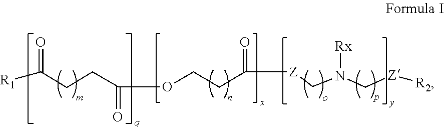
wherein,
for Formula I, n is an integer from 1-30, m, o, and p are independently integers from 1-20, x, y, and q are independently integers from 1-1000, Rₓ is hydrogen, substituted or unsubstituted alkyl, or substituted or unsubstituted aryl, or substituted or unsubstituted alkoxy, Z and Z′ are independently O or NR′, wherein R′ is hydrogen, substituted or unsubstituted alkyl, or substituted or unsubstituted aryl, R₁ and R₂ are independently absent or are chemical entities containing a hydroxyl group, a primary amine group, a secondary amine group, a tertiary amine group, or combinations thereof, and
(ii) a surfactant on the surface of the nanoparticle, non-covalently conjugated to the polymer having a structure of Formula I.

19. The nanoparticles of claim 18, wherein for Formula I, R₁ and/or R₂ are not

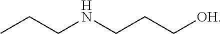

20. The nanoparticles of claim 18, wherein Formula I has a structure: 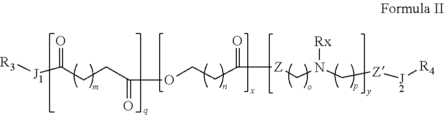
wherein J₁ and J₂ are independently linking moieties or absent,
R₃ and R₄ are independently substituted alkyl containing a hydroxyl group, a primary amine group, a secondary amine group, a tertiary amine group, or combinations thereof.

21. The nanoparticles of claim 18, wherein Formula I has a structure:

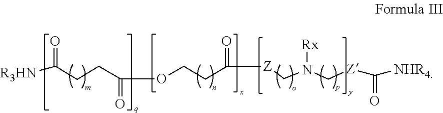

22. The nanoparticles of claim 18, wherein for Formula I n is 4, 10, 13, or 14.

23. The nanoparticles of claim 18, wherein for Formula I Rₓ is substituted or unsubstituted alkyl.

24. The nanoparticles of claim 18, comprising a second PACE polymer, wherein the second PACE polymer has a weight average molecular weight between about 2,000 Daltons and about 30,000 Daltons, between about 2,000 Daltons and about 25,000 Daltons, between about 2,000 Daltons and about 20,000 Daltons, or between about 5,000 Daltons and about 10,000 Daltons.

25. The nanoparticles of claim 20, wherein R₃ and/or R₄ are independently selected from the group consisting of

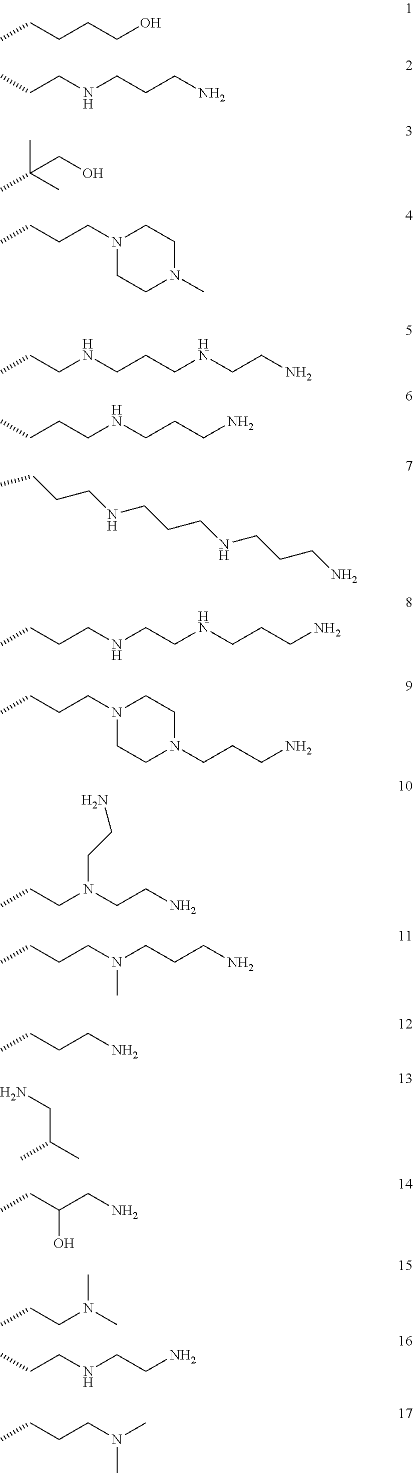

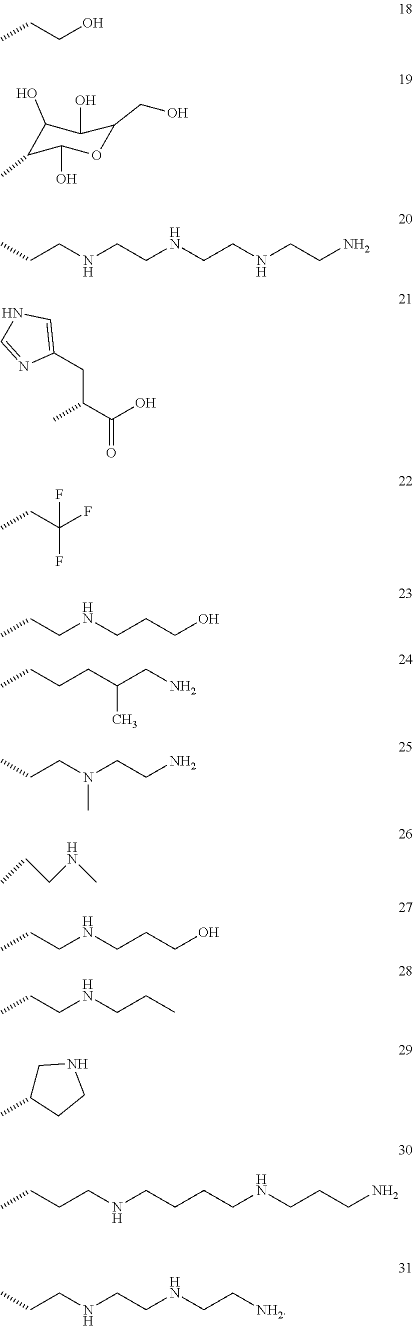

26. The nanoparticles of claim 18, wherein the surfactant contains a structure: 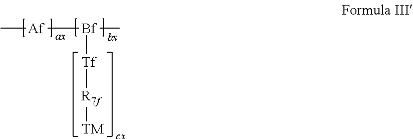
wherein:
each Af is independently a hydrophobic or hydrophilic monomer residue,
Bf is substituted alkyl, substituted aryl, substituted cycloalkyl, substituted heterocyclyl, substituted amine, substituted carbonyl, substituted amide, or substituted sulfone,
wherein Tf is independently absent, oxygen, sulfur, alkyl, substituted alkyl, amide, substituted amide, amine, substituted amine, carbonyl, substituted carbonyl,
wherein R_{7f} is independently hydrogen, alkyl, substituted alkyl, amide, substituted amide, substituted sulfone, unsubstituted sulfone, aryl, substituted aryl, cycloalkyl, substituted cycloalkyl, maleimide, amine, substituted amine, thiol, N-hydroxysuccinimide ester, succinimide, azide, acrylate, methacrylate, alkyne, hydroxide, or isocynate,
TM is a targeting moiety,
ax is independently an integer between 0 and 10,000, between 0 and 5,000, or between 0 and 1,000, and
bx and cx are independently an integer between 1 and 10,000, between 1 and 5000, or between 1 and 1,000.

27. The nanoparticles of claim 18, wherein the surfactant contains a structure:

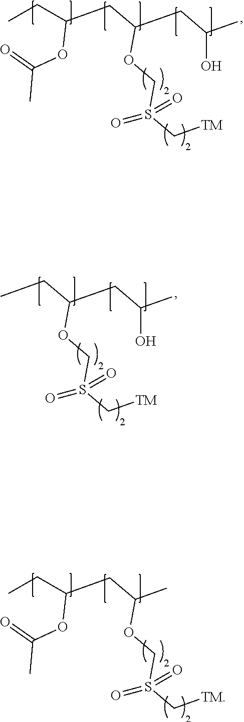

28. The nanoparticles of claim 1, comprising therapeutic agents, prophylactic agents, diagnostic agents, or a combination thereof.

29. The nanoparticles of claim 1, comprising nucleic acids.

30. The nanoparticles of claim 18, comprising nucleic acids.
